(19) United States
(12) Patent Application Publication
(10) Pub. No.: US 2025/0210197 A1
(43) Pub. Date: Jun. 26, 2025

(54) MACHINE LEARNING FOR DIFFERENTIATING AMONG MULTIPLE DISEASES

(71) Applicant: The Chinese University of Hong Kong, Shatin (HK)

(72) Inventors: Siew Chien Ng, Hong Kong SAR (CN); Ka Leung Chan, Tai Po (CN); Qin Liu, Shenyang City (CN); Qi Su, Tai Po (CN)

(21) Appl. No.: 19/074,303

(22) Filed: Mar. 7, 2025

Related U.S. Application Data
(63) Continuation of application No. PCT/CN2023/116786, filed on Sep. 9, 2023.
(60) Provisional application No. 63/405,311, filed on Sep. 9, 2022.

(51) Int. Cl. G16H 50/20; G16B 40/00

(52) U.S. Cl. CPC G16H 50/20; G16B 40/00

(57) ABSTRACT

This disclosure provides a predictive risk assessment tools to determine personalized risk of multiple diseases in a subject using microbiome. Current risk prediction test using microbiome may only detect one disease or health condition at a time. By determining multiple diseases simultaneously, the disclosed techniques can provide a cost-effective method to support clinical decision making, and hence to help improve disease prevention and management.

### CROSS-REFERENCES TO RELATED APPLICATION

This application is a continuation application of International Patent Application No. PCT/CN2023/116786 entitled “MACHINE LEARNING FOR DIFFERENTIATING AMONG MULTIPLE DISEASES,” filed on Sep. 4, 2023, which is a PCT of and claims the benefit of U.S. Provisional Application No. 63/405,311 entitled “MACHINE LEARNING FOR DIFFERENTIATING AMONG MULTIPLE DISEASES,” filed on Sep. 9, 2022, each of which are herein incorporated by reference in their entirety for all purposes.

### BACKGROUND

Fecal microbiome-based analyses can represent non-invasive approaches for detecting human diseases but may be limited by shared microbial signals across different disease phenotypes. Existing risk prediction model using metagenomics data may only be able differentiate diseases dichotomously. Even when reference data of multi-diseases are available, the risk of each disease may be determined one at a time. Such risk prediction models can involve drawing data of subjects with the first disease and data of healthy controls from a database. The database data can then be compared with data from the test subjects. The process can be repeated by drawing data of subjects with the second disease and data of healthy control, then comparing these data with the data from test subjects. Accordingly, a method to determine risk of multi-diseases simultaneously is desirable.

### BRIEF SUMMARY

This disclosure provides techniques for predictive risk assessment tools to determine personalized risk of multiple diseases in a subject using microbiome. Current risk prediction test using microbiome may only detect one disease or health condition at a time. By determining multiple diseases simultaneously, the disclosed techniques can provide a cost-effective method to support clinical decision making, and hence to help improve disease prevention and management.

The techniques include generating a training data set for subjects having a plurality of known health classifications. For each subject cohort: the relative abundance of DNA fragments can be measured for each bacterial species, and the bacterial species can correspond to the bacterial species in a fecal sample of each of the subjects. Each subject in a cohort can have a health classification where each cohort corresponds to a health classification including healthy and a plurality of conditions. At least ten of the bacterial species were present in greater than a specified percentage (e.g., at least 5%) of the subjects. A feature vector containing the relative abundance for the bacterial species can be generated for each subject. The training data can be used to train a multi-class machine learning model. The training data can include the known health classifications for the subjects and the feature vectors for the subjects. The multi-class machine learning model provide a probability for each of the M health classifications. The training optimizes sensitivity and specificity for determining a correct health condition by achieving a highest average AUC of the M health classifications.

These and other embodiments of the disclosure are described in detail below. For example, other embodiments are directed to systems, devices, and computer readable media associated with methods described herein.

### BRIEF DESCRIPTION OF THE DRAWINGS

FIG. 1 is a diagram of a framework for dataset partition, model training and independent validation according to an embodiment.

FIG. 2A is a flowchart showing an overview of generating a multi-class machine learning model according to an embodiment.

FIG. 2B is a flowchart showing techniques for applying the model to predict risk of pre-determined health conditions in a subject according to an embodiment.

FIG. 3A is a table showing a summary of the demographics of the individuals providing fecal samples to an embodiment.

FIG. 3B is a graph showing the alpha diversity of bacteria in the cohort according to an embodiment.

FIG. 3C is a graph showing the richness of fecal biome bacteria in the cohort according to an embodiment.

FIG. 3D is a graph showing the association of fecal biome bacterial species to the health condition phenotypes according to an embodiment.

FIGS. 4A-4C show fecal microbiome differences among health conditions according to an embodiment.

FIG. 5A shows a graph of a receiver operating characteristic (ROC) curve for the random forest (RF) multi-class classifier according to an embodiment.

FIG. 5B shows a chart of thresholds at the highest Youden index for the random forest (RF) multi-class classifier according to an embodiment.

FIG. 6A-6D show a charts with the area under the receiver operating characteristic curve (AUC) and area under the precision-recall curve (AUPR) for models trained to classify health conditions according to an embodiment.

FIG. 7A-7I show graphs of the distribution of probabilities yielded by the trained random forest (RF) multi-class classifier according to an embodiment.

FIGS. 8A-8B show the performance of the random forest (RF) multi-class classifier in one versus one discrimination of multiple health conditions according to an embodiment.

FIGS. 9A-9B are graphs showing the performance of random forest (RF) multi-class classifier stratified by age.

FIGS. 10A-10D show the performance of a random forest (RF) multi-class classifier that was trained and tested on a balanced cohort size according to an embodiment.

FIG. 11A-11B show the performance of a random forest (RF) multi-class classifier using different numbers of features.

FIGS. 12A-12D show independent validations of the performance of the random forest (RF) multi-class classifier according to an embodiment.

FIG. 13A-13C show the performance of the random forest (RF) multi-class classifier using the top 50 features according to an embodiment.

FIG. 14 is a chart chowing microbial species associated with health status or different health conditions according to an embodiment.

FIG. 15 is a diagram of a method for training a model to predict health conditions according to an embodiment.

FIG. 16 is a diagram of a method for predicting health conditions according to an embodiment.

FIG. 17 shows a block diagram of a computer system according to embodiments of the present disclosure.

FIG. 18 illustrates a measurement system according to an embodiment of the present disclosure.

An appendix shows the following tables.

Table 1 shows a list of 325 bacterial species according to an embodiment.

Table 2 shows a list of 50 bacterial species according to an embodiment.

Table 3 shows a list of 10 bacterial species according to an embodiment.

Table 4 shows a list of bacteria that can be used to treat diseases according to an embodiment.

### TERMS

The terms “sample” or “biological sample” refer to any sample that is taken from a subject suspected of having a health condition and contains one or more nucleic acid molecule(s) of interest. The biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), intraocular fluids (e.g., the aqueous humor), etc. Stool (fecal) samples can also be used. In various embodiments, the majority of DNA in a biological sample that has been enriched for cell-free DNA (e.g., a plasma sample obtained via a centrifugation protocol) can be cell-free, e.g., greater than 50%, 60%, 70%, 80%, 90%, 95%, or 99% of the DNA can be cell-free. A centrifugation protocol for enriching cell-free DNA from a biological sample can include, for example, centrifuging the biological sample at 3,000 g×10 minutes, obtaining the fluid part of the centrifuged sample, and re-centrifuging at for example, 30,000 g for another 10 minutes to remove residual cells. As part of an analysis of a biological sample, a statistically significant number of cell-free DNA molecules can be analyzed (e.g., to provide an accurate measurement) for a biological sample. In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed. At least a same number of sequence reads can be analyzed.

The terms “reference” and “reference genome” refers to generally refers to a haploid or diploid genome to which sequence reads from the biological sample can be aligned and compared. For a haploid genome, there is only one nucleotide at each locus. For a diploid genome, heterozygous loci can be identified, with such a locus having two alleles, where either allele can allow a match for alignment to the locus. A reference genome can be a reference microbe genome, metagenomic assembled genomes, or species-level genome bins that corresponds to a particular microbe species, e.g., by including one or more microbe genomes, metagenomic assembled genomes, or species-level genome bins.

The phrase “healthy”, “control”, and “normal” may be interchangeably used to generally refers to a subject possessing good health. Such a subject demonstrates an absence of any health condition or disease. A “healthy individual” may have other diseases or conditions, unrelated to the condition being assayed, that may normally not be considered “healthy”.

The term “fragment” (e.g., a DNA or an RNA fragment), as used herein, can refer to a portion of a polynucleotide or polypeptide sequence that comprises at least 3 consecutive nucleotides. A nucleic acid fragment can retain the biological activity and/or some characteristics of the parent polypeptide. A nucleic acid fragment can be double-stranded or single-stranded, methylated or unmethylated, intact or nicked, complexed or not complexed with other macromolecules, e.g. lipid particles, proteins. A nucleic acid fragment can be a linear fragment or a circular fragment. A bacterial nucleic acid can refer to any nucleic acid of bacteria. Such a bacterial nucleic acid may be released from a microorganism. As part of an analysis of a biological sample, a statistically significant number of fragments can be analyzed, e.g., at least 1,000 fragments can be analyzed. As other examples, at least 5,000, 10,000 or 50,000 or 100,000 or 500,000 or 1,000,000 or 5,000,000 fragments, or more, can be analyzed.

The term “assay” generally refers to a technique for determining a property of a nucleic acid or a sample of nucleic acids (e.g., a statistically significant number of nucleic acids or relative abundance of microorganisms), as well as a property of the subject from which the sample was obtained. An assay may include a technique for determining the quantity of nucleic acids in a sample, genomic identity of nucleic acids in a sample, the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art may be used to detect any of the properties of nucleic acids mentioned herein. Properties of nucleic acids include a sequence, quantity, genomic identity, copy number, a methylation state at one or more nucleotide positions, a size of the nucleic acid, a mutation in the nucleic acid at one or more nucleotide positions, and the pattern of fragmentation of a nucleic acid (e.g., the nucleotide position(s) at which a nucleic acid fragments). The term “assay” may be used interchangeably with the term “method”. An assay or method can have a particular sensitivity and/or specificity (e.g., based on selection of one or more cutoff values), and their relative usefulness as a diagnostic tool can be measured using Receiver Operating Characteristic (ROC) Area-Under-the-Curve (AUC) statistics.

A “sequence read” refers to a string of nucleotides obtained from any part or all of a nucleic acid molecule. For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes as may be used in microarrays, or amplification techniques, such as the polymerase chain reaction (PCR) or linear amplification using a single primer or isothermal amplification. Example sequencing techniques include shotgun metagenomic sequencing, 16S rRNA sequencing, massively parallel sequencing, targeted sequencing, Sanger sequencing, sequencing by ligation, ion semiconductor sequencing, and single molecule sequencing (e.g., using a nanopore, or single-molecule real-time sequencing (e.g., from Pacific Biosciences)). Such sequencing can be random sequencing or targeted sequencing (e.g., by using capture probes hybridizing to specific regions or by amplifying certain region, both of which enrich such regions). Example PCR techniques include real-time PCR and digital PCR (e.g., droplet digital PCR). As part of an analysis of a biological sample, a statistically significant number of sequence reads can be analyzed, e.g., at least 1,000 sequence reads can be analyzed. As other examples, at least 5,000, 10,000 or 50,000 or 100,000 or 500,000 or 1,000,000 or 5,000,000 sequence reads, or more, can be analyzed.

The term “classification” as used herein refers to any number(s) or other characters(s) that are associated with a particular property of a sample. For example, a “+” symbol (or the word “positive”) could signify that a sample has a particular health classification, where a vector of such symbols can be provided, each indicating a classification for a corresponding healthy condition (e.g., healthy or a plurality of unhealthy conditions). A classification can be binary (e.g., positive or negative) or have more levels of classification (e.g., a scale from 1 to 10 or 0 to 1), including probabilities.

The term “parameter” as used herein means a numerical value that characterizes a quantitative data set and/or a numerical relationship between quantitative data sets. For example, a ratio (or function of a ratio) between a first amount of a first nucleic acid sequence and a second amount of a second nucleic acid sequence is a parameter. The parameter can be used to determine any classification described herein.

A “relative abundance” may refer to a proportion (e.g., a percentage, fraction, or concentration). In particular, a relative abundance of a particular bacterial species can provide a proportion of the bacterial DNA fragments that are from the particular bacterial species (e.g., as determined by aligning a sequence read or via a probe specified to that particular bacterial species). The relative abundance of a particular bacterial species can be determined by dividing the raw abundances of that particular species by the total number of counts of species per sample.

The terms “cutoff” and “threshold” refer to predetermined numbers used in an operation. For example, a cutoff size can refer to a size above which fragments are excluded. A threshold value may be a value above or below which a particular classification applies. Either of these terms can be used in either of these contexts. A cutoff or threshold may be “a reference value” or derived from a reference value that is representative of a particular classification or discriminates between two or more classifications. A cutoff may be predetermined with or without reference to the characteristics of the sample or the subject. For example, cutoffs may be chosen based on the age or sex of the tested subject. A cutoff may be chosen after and based on output of the test data. For example, certain cutoffs may be used when the sequencing of a sample reaches a certain depth. As another example, reference subjects with known classifications of one or more conditions and measured characteristic values (e.g., a relative abundance, a statistical size value, or a count) can be used to determine reference levels to discriminate between the different conditions and/or classifications of a condition (e.g., whether the subject has the condition). A reference value can be selected as representative of one classification (e.g., a mean) or a value that is between two clusters of the metrics (e.g., chosen to obtain a desired sensitivity and specificity). As another example, a reference value can be determined based on statistical simulations of samples. Any of these terms can be used in any of these contexts. Such a reference value can be determined in various ways, as will be appreciated by the skilled person. For example, metrics can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples. A particular value for a cutoff, threshold, reference, etc. can be determined based on a desired accuracy (e.g., a sensitivity and specificity).

A “health classification” refers to a classification of a subject's health. As examples, a health classification can be provided as whether a subject is healthy or has a condition, e.g., conditions mentioned below and herein. A classification can be provided for each of a plurality of conditions or whether the subject is healthy. As examples, the classification can be a binary value as to whether a condition is present or a probability value that a condition is present.

A “level of a condition” can refer to the presence or absence, or an amount of bacteria present in a biological sample. For example, the level of a condition can indicate a number of sequence reads associated with bacteria (e.g., reads per million) that are obtained from a sample (e.g., a fecal sample) of a subject. The presence of bacteria can indicative the amount, degree, or severity of a condition associated with a bacterium. In some instances, the amount, degree, or severity of conditions is predicted based on the amount of bacteria in the biological sample. The level of the condition may be a number or other indicia, such as symbols, alphabet letters, and colors. The level may be zero. The level of the condition can be used in various ways. Example conditions include gastrointestinal (GI) diseases such as colorectal cancer (CRC), colorectal adenomas (CA), Crohn's disease (CD), ulcerative colitis (UC), and inflammatory bowel disease (IBD); obesity; and cardiovascular disease (CVD).

The term “true positive” (TP) can refer to subjects having a condition. True positive generally refers to subjects that have a disease (e.g., post-acute COVID-19 syndrome). True positive generally refers to subjects having a condition and are identified as having the condition by an assay or method of the present disclosure.

The term “true negative” (TN) can refer to subjects that do not have a condition or do not have a detectable condition. True negative generally refers to subjects that do not have a disease or a detectable disease, including post-acute COVID-19 syndrome. True negative generally refers to subjects that do not have a condition or do not have a detectable condition or are identified as not having the condition by an assay or method of the present disclosure.

The term “false positive” (FP) can refer to subjects not having a condition. False positive generally refers to subjects not having a disease. The term false positive generally refers to subjects not having a condition but are identified as having the condition by an assay or method of the present disclosure.

The term “false negative” (FN) can refer to subjects that have a condition. False negative generally refers to subjects that have a disease. The term false negative generally refers to subjects that have a condition but are identified as not having the condition by an assay or method of the present disclosure.

The terms “sensitivity” or “true positive rate” (TPR) can refer to the number of true positives divided by the sum of the number of true positives and false negatives. Sensitivity may characterize the ability of an assay or method to correctly identify a proportion of the population that truly has a condition. For example, sensitivity may characterize the ability of a method to correctly identify the number of subjects within a population having a disease. In another example, sensitivity may characterize the ability of a method to correctly identify one or more markers indicative of a disease.

The terms “specificity” or “true negative rate” (TNR) can refer to the number of true negatives divided by the sum of the number of true negatives and false positives. Specificity may characterize the ability of an assay or method to correctly identify a proportion of the population that truly does not have a condition. For example, specificity may characterize the ability of a method to correctly identify the number of subjects within a population not having a disease. In another example, specificity may characterize the ability of a method to correctly identify one or more markers indicative of a disease.

The term “ROC” or “ROC curve” can refer to the receiver operator characteristic curve. The ROC curve can be a graphical representation of the performance of a binary classifier system. For any given method, an ROC curve may be generated by plotting the sensitivity against the specificity at various threshold settings. The sensitivity and specificity of a method for predicting a level of health condition in a subject may be determined at various probability generated by a machine learning model based on the concentrations of microbial DNA in the fecal sample of the subject. Furthermore, provided at least one of the three parameters (e.g., sensitivity, specificity, and the threshold setting), and ROC curve may determine the value or expected value for any unknown parameter. The unknown parameter may be determined using a curve fitted to the ROC curve. The term “AUC” or “ROC-AUC” generally refers to the area under a receiver operator characteristic curve. This metric can provide a measure of diagnostic utility of a method, taking into account both the sensitivity and specificity of the method. Generally, ROC-AUC ranges from 0.5 to 1.0, where a value closer to 0.5 indicates the method has limited diagnostic utility (e.g., lower sensitivity and/or specificity) and a value closer to 1.0 indicates the method has greater diagnostic utility (e.g., higher sensitivity and/or specificity). See, e.g., Pepe et al, “Limitations of the Odds Ratio in Gauging the Performance of a Diagnostic, Prognostic, or Screening Marker,” Am. J. Epidemiol 2004, 159 (9): 882-890, which is entirely incorporated herein by reference. Additional approaches for characterizing diagnostic utility using likelihood functions, odds ratios, information theory, predictive values, calibration (including goodness-of-fit), and reclassification measurements are summarized according to Cook, “Use and Misuse of the Receiver Operating Characteristic Curve in Risk Prediction,” Circulation 2007, 115:928-935, which is entirely incorporated herein by reference.

“Negative predictive value” or “NPV” may be calculated by TN/(TN+FN) or the true negative fraction of all negative test results. Negative predictive value is inherently impacted by the prevalence of a condition in a population and pre-test probability of the population intended to be tested. “Positive predictive value” or “PPV” may be calculated by TP/(TP+FP) or the true positive fraction of all positive test results. It is inherently impacted by the prevalence of the disease and pre-test probability of the population intended to be tested. See, e.g., O'Marcaigh A S, Jacobson R M, “Estimating The Predictive Value Of A Diagnostic Test, How To Prevent Misleading Or Confusing Results,” Clin. Ped. 1993, 32 (8): 485-491, which is entirely incorporated herein by reference.

A “machine learning model” (ML model) can refer to a software module configured to be run on one or more processors to provide a classification or numerical value of a property of one or more samples. An ML model can be generated using sample data (e.g., training data) to make predictions on test data. One example is an unsupervised learning model. Another example type of model is supervised learning that can be used with embodiments of the present disclosure. Example supervised learning models may include different approaches and algorithms including analytical learning, statistical models, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbor algorithm, probably approximately correct learning (PAC) learning, ripple down rules, a knowledge acquisition methodology, symbolic machine learning algorithms, subsymbolic machine learning algorithms, minimum complexity machines (MCM), random forests, ensembles of classifiers, ordinal classification, data pre-processing, handling imbalanced datasets, statistical relational learning, or Proaftn, a multicriteria classification algorithm. The model may include linear regression, logistic regression, deep recurrent neural network (e.g., long short term memory, LSTM), hidden Markov model (HMM), linear discriminant analysis (LDA), k-means clustering, density-based spatial clustering of applications with noise (DBSCAN), random forest algorithm, support vector machine (SVM), or any model described herein. Supervised learning models can be trained in various ways using various cost/loss functions that define the error from the known label (e.g., least squares and absolute difference from known classification) and various optimization techniques, e.g., using backpropagation, steepest descent, conjugate gradient, and Newton and quasi-Newton techniques.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and more preferably within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

Standard abbreviations may be used, e.g., bp, base pair(s); kb, kilobase(s); pi, picoliter(s); s or sec, second(s); min, minute(s); h or hr, hour(s); aa, amino acid(s); nt, nucleotide(s); and the like.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the embodiments of the present disclosure, some potential and exemplary methods and materials may now be described.

### DETAILED DESCRIPTION

This disclosure provides techniques for predictive risk assessment tools to determine personalized risk of multiple diseases in a subject using microbiome. Current risk prediction test using microbiome may only detect one disease or health condition at a time. By determining multiple diseases simultaneously, the disclosed techniques can provide a cost-effective method to support clinical decision making, and hence to help improve disease prevention and management.

Recent studies have shown that imbalanced intestinal microbiota, termed “dysbiosis” contributes to various human diseases [1], but dysbiosis' potential role in diseases prediction is largely unexplored due to limited sample size, insufficient validation, and wide heterogeneity across studies [2,3]. Also, current development of microbiome markers has mostly used binary classifiers (presence or absence of disease) and focused on gastrointestinal (GI) diseases such as colorectal cancer (CRC) and inflammatory bowel disease (IBD) [4-6].

Emerging evidence however suggest that the gut microbiome is involved in pathogenesis of non-GI diseases, including obesity and cardiovascular disease (CVD) [7,8]. Recently, coronavirus disease 2019 (COVID-19) and its long-term sequelae known as post-acute COVID-19 syndrome (PACS) were also associated with significant and persistent gut dysbiosis [9,10,11]. Given that most health condition phenotypes (e.g., corresponding to a health classification) exhibit overlapping gut microbiome signatures [12], single-disease diagnostic models (single health condition phenotype versus health control) are likely to be confounded by signals shared across unrelated health condition phenotypes and may lead to misclassification.

This disclosure provides techniques for simultaneously predicting risk of multiple health condition phenotypes in a subject using a multi-class machine learning model. The health conditions can include post-acute COVID-19 syndrome (PACS or Long COVID), Crohn's disease (CD), colorectal cancer (CRC), colorectal adenoma (CA), obesity (Ob), irritable bowel syndrome (IBS), ulcerative colitis (UC) and cardiovascular disease (CVD). Metagenomics data, such as fecal microbiome datasets for multiple human health conditions, can be used to train a machine learning multi-class model. Existing risk prediction models using metagenomics data may differentiate diseases dichotomously. However, with dichotomous techniques, the risk of each disease may have to be determined one at a time even when reference data of multi-diseases are available. The techniques in this disclosure provide a method to determine risk of multi-diseases simultaneously.

Metagenomics can include the study of genetic material from a community of organisms. The communities can be found in samples include fecal samples, gut mucosa samples, saliva, skin swabs, soil samples, water samples, and the like. For example, species characterization for a fecal sample can be used to identify health condition phenotypes. Various techniques, such as shotgun metagenomic sequencing, can be used to characterize a sample's species. In shotgun metagenomic sequencing, DNA is obtained from a heterogenous sample of cells and segmented into DNA fragments that can be aligned with the genomes of multiple microbial species to identify species in the sample.

Techniques in this disclosure include machine learning models that were trained using a dataset derived from a cohort comprising 2,320 individuals with nine well-characterized health condition phenotypes. Fecal samples from this cohort provided the DNA used to characterize the microbial species in the dataset. The sequencing produced 14.6 terabytes of sequenced deoxyribonucleic acid (DNA) for a cohort of 2,320 individuals. The health condition phenotypes (e.g., health conditions) include CRC, CA, CD, Ob, UC, IBS, PCAS, and healthy control phenotype. Fecal samples from the cohort were used to produce a processed data set containing the microbial species that were present in over 5% of individuals in the cohort. The processed dataset can be used training data for a machine learning model that can use sequenced fecal samples to classify individuals based on their health condition phenotypes.

A classifier can be a model that outputs a probability (example of a health classification) that a given entity is in a class. The probability can be a value between 0.0 and 1.0 and a value, called the discrimination threshold, can be the dividing line between classes. For example, if the discrimination threshold is 0.6, an entity with a probability between 0.0 and 0.6 can be sorted into class A and if the probability is between 0.6 and 1.0 the entity can be sorted into class B.

The performance of a classification model (e.g., a classifier) can be evaluated using a plot called a receiver operating characteristic (ROC) curve. The plot shows the classifier's ability to sort entities into classes as the discrimination threshold is varied. The y-axis for the plot is the true positive rate, or sensitivity, and the x-axis is the false positive rate (e.g., 1-sensitivity). The area under the ROC (AUC) is a measure of a classification model's performance. The AUC value indicates the classifier's separability and the value can range from 0.0 to 1.0 with a higher value indicating higher separability. For example, a classifier with an AUC of 1.0 is able to sort all entities into the correct classes.

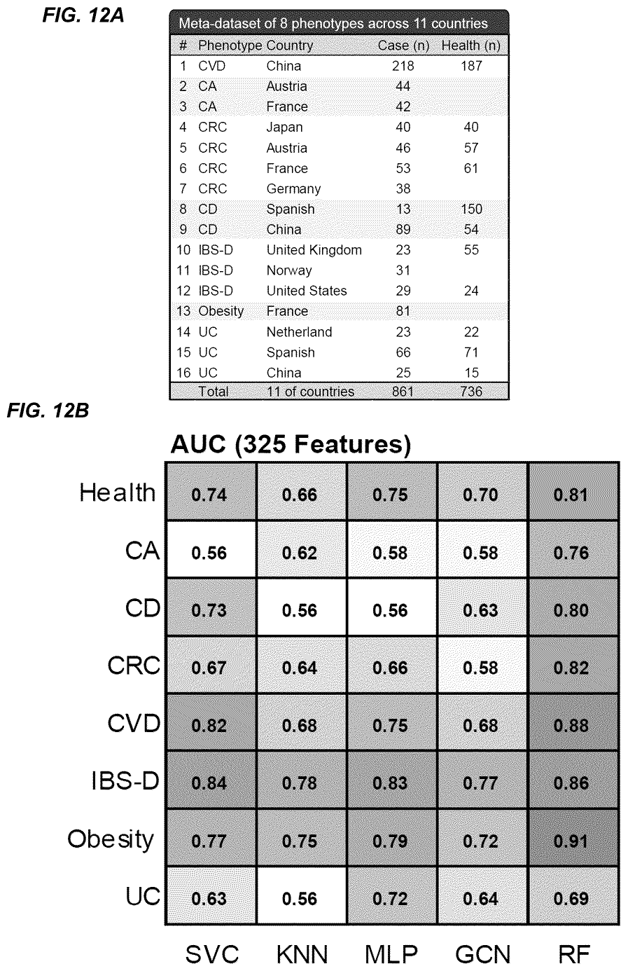

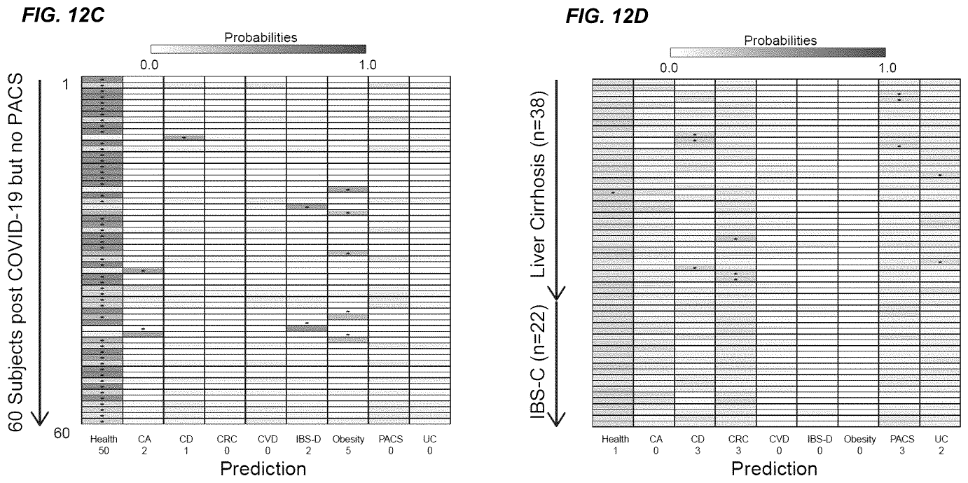

Using the techniques in this disclosure, a trained machine-learning multi-class classifier achieved area under the receiver operating characteristic curve (AUC) of 0.90 to 0.99 (Interquartile range, IQR, 0.91-0.94) for health condition phenotype prediction, with high sensitivities (IQR, 0.87-0.93) and specificities (IQR 0.83-0.95). The classifier remained predictive (average AUC 0.82, IQR 0.79-0.87, FIG. 12B) in cross-regional public datasets. We correlated top 50 microbial species contributing to the model and disease phenotypes, thereby identified health-condition-phenotype-specific microbial signatures.

### I. Multi Class Machine Learning Model

Multi class classification is the problem of classifying entities into multiple categories. An algorithm can be trained into a model that can perform multi class classification using training data with known classifications. During training, the training data is input to the algorithm and the algorithm's parameters are modified until the classification output by the algorithm matches the known classification.

### A. Model Selection

Classification tasks in machine learning involving more than two classes can be known as “multi-class classification”, which can effectively account for the confounding effects of coexisting classes [16]. Because of the gut biome's complexity, multi-class classification can be better for the development of microbiome-based diagnostic tools than binary classifiers (single disease versus control). For example, based on our cohort of 2,320 individuals representing nine health conditions, including eight diseases and a healthy group, we trained five machine learning multi-class classifiers to classify different diseases using normalized data at the species level (325 species). The dataset was divided into a training set and a test set. The training set was used to train the models and the performance of the trained models was assessed with the withheld test set.

Multi-class classification can be performed by a machine learning model that can be trained from an algorithm. Training data can be input into the algorithm as feature vectors and the algorithm can output a classification based on the input. Feature vectors can be n-dimensional vectors containing numerical characteristics for an entity. The training data can have a known classification, and, during training, the algorithm's parameters can be modified until the output classification matches the known classification. For example, the training data can include the microbiome from a cohort member's stool sample and the classification can be a phenotype (e.g., presence or absence of a health condition).

The multi-class classification model can be trained from several algorithms. For instance, K nearest neighbors is an algorithm that can maintain existing examples and categorize new examples using a similarity metric (e.g., distance functions). The Support Vector Machine, or SVM, is a common Supervised Learning technique and the algorithms used to create a SVM may be used to solve both classification and regression issues. The SVM algorithm classifies data points by finding the optimum line or decision boundary within a n-dimensional space. The decision boundary separates the data point into classes so that additional data points may be readily placed in the proper category in the future. A hyperplane can be a name for the optimal choice boundary. Random forest is a supervised learning approach used in machine learning for classification and regression. Random forest is a supervised machine learning algorithm that averages the results of many decision trees applied to distinct subsets of a dataset to improve the dataset's projected accuracy. MLP Classifier stands for multi-layer perceptron classifier which is a feedforward artificial neural network. The MLP classifier consists of nodes connected by edges with the nodes arranged in layers. Like the SVM algorithm, the MLP algorithm can classify data by finding an optimized decision boundary in a n-dimensional space. Graph convolutional neural network (GCN) is an approach for semi-supervised learning on graph-structured data. GCN is based on a variant of convolutional neural networks which can operate directly on graphs. The choice of convolutional architecture can be motivated by a localized first-order approximation of spectral graph convolutions. The number of graph edges can scale linearly and the model can learn hidden layer representations that can encode local graph structure and features of nodes.

### B. Model Training and Validation

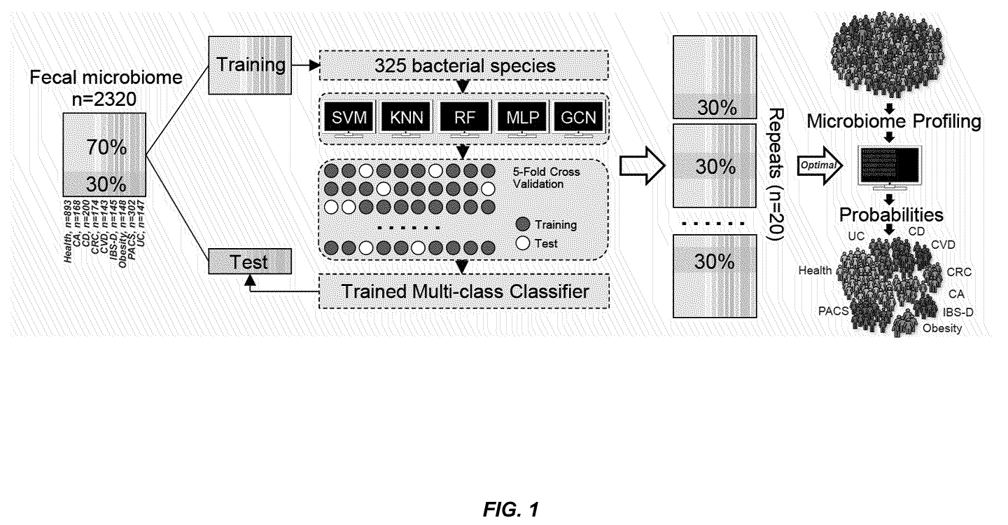

FIG. 1 is a diagram of a framework for dataset partition, model training and independent validation according to an embodiment. The machine learning model can be trained using a metagenomics dataset derived from subjects with one or more known health conditions. The health conditions can include healthy controls that have not been diagnosed with a known condition.

Metagenomics dataset can be an aggregate set of metagenomics data that can be obtained by sequencing a sample containing genetic material from a community of organisms. For example, the metagenomics dataset can be a set of fecal samples collected or downloaded from public database. The metagenomics dataset can include sequencing data generated from the same standardized protocol including steps from fecal DNA extraction to sequencing, raw data quality filter, host reads decontamination, to microbiome interpretation. The metagenomics data for an individual whose sample being tested (e.g., classified) by the trained model can use the same standardized protocol used to generate the model training data.

The health condition of subjects whose samples are included in the training dataset for the multi-class classifier can be determined by formal diagnosis specific a condition. For example, (1) PACS is defined as at least one persistent symptom or long-term complications (only appeared after COVID-19) of SARS-COV-2 infection beyond 4 weeks from the onset of symptoms which could not be explained by an alternative diagnosis; (2) CVD was confirmed by examining cardiovascular stenosis and plaque by carotid ultrasounds; (3) IBS subjects were diagnosed according to ROME III criteria. Enteroscopy can be performed to exclude any GI disorders presenting bowel habit change, such as inflammatory bowel disease, Coceliac disease, parasite infestations, or other organic disorders; (4) CD was diagnosed by endoscopy, radiology, and histology examinations; (5) UC was defined by endoscopy, radiology, and histology; (6) CRC and CA was diagnosed by colonoscopy and histology examinations. (7) Obese was defined by BMI over 28, and the recruit subjects had no other diseases.

Subjects with cardiovascular disease (CVD) were recruited from the public as part of a survey of cardiovascular health in the Hong Kong general population. Subjects underwent carotid ultrasounds to measure intima-media thickness (IMT) of the common, internal, external carotid arteries (CCA, ICA and ECA, respectively) and carotid bulbs and subjects that had ≥50% stenosis in a single or multiple vessels were regarded as having the risk of CVD.

Individuals diagnosed with each health condition can provide samples that can be sequenced to produce a metagenomics dataset. For example, fecal samples, or gut mucosal samples, can be sequenced to determine the relative abundance of all species in the microbiota. Gut mucosal samples can be obtained through a biopsy of the gastrointestinal tract, sampling using a luminal brush, catheter aspiration of the bowel, surgical resection of the gastrointestinal tract, and the like. Although in some cases, not all species need to be used. The number of individuals can be 10, 50, 100, 200, 300, 400, 500, 600, 700, 800, 900, 1000, 1250, 1500, 1750, 2000, 2500, 3000, 4000, 5000, 6000, 7000, 8000, 9000, 10000, 15000, 20000, 50000, 100000 500000, or 1000000 individuals. Feature vectors can be created from the metagenomics dataset to produce a training dataset. The feature vector can be a N-dimensional vector where N is a number of bacterial species. N can be the number of bacterial species detected in the metagenomics dataset, the top 350 bacterial species in the dataset, the top 50 bacterial species in the dataset, or all species in the dataset a prevalence that is greater than 0.15%.

A nested cross-validation procedure can be applied to calculate within-cohort accuracy by randomly splitting the feature vectors into training data and test data. The training data can be randomly divided into k subsets. The subsets can be used to create k folds where k−1 of the subsets are used as training sets and the remaining subset is used as a within-training validation set. The training sets can be used to train the model and the within-training validation set to evaluate the model's performance during training. The test data, which may not be used to train the model, can be used to evaluate the trained model.

For example, the validation procedure can be a 20-times repeated, fivefold-stratified cross-validation (balancing class proportions across folds) where k=5. For each split in this example, an L1-regularized (Lasso) logistic regression model can be trained on the training sets, which can then be used to predict classifications for the validation set. The model can be evaluated on the test data after this process has been repeated 20 times. The lambda hyperparameter can be selected for each model to maximize the AUC-ROC under the constraint that the model contained at least five nonzero coefficients. The lambda hyperparameter for a model can be varied until the maximum AUC-ROC for that model is found.

The area under the receiver operating characteristic curve (AUC) can be used to compare the performance across models of different methods and features. The AUC is a widely applied metric that considers the trade-offs between sensitivity and specificity at all possible thresholds for comparing the performance across various classifiers. The baseline AUC value can be 0.5 for a random classifier.

The area under the precision-recall curve (AUPR) can be provided as a complimentary assessment, which considers the trade-offs between precision (or positive predictive value) and recall (or sensitivity) with a baseline that equals the proportion of positive disease cases in all samples. Precision is the fraction of relevant instances among the retrieved instances and the recall is the total proportion of relevant instance that were retrieved. The precision recall curve is a plot of the precision (x-axis) vs recall (y-axis). An AUPR value of 1.0 indicates high precision and recall while a value of 0.0 indicates low precision and recall.

### C. Generating a Multi-Class Machine Learning Model

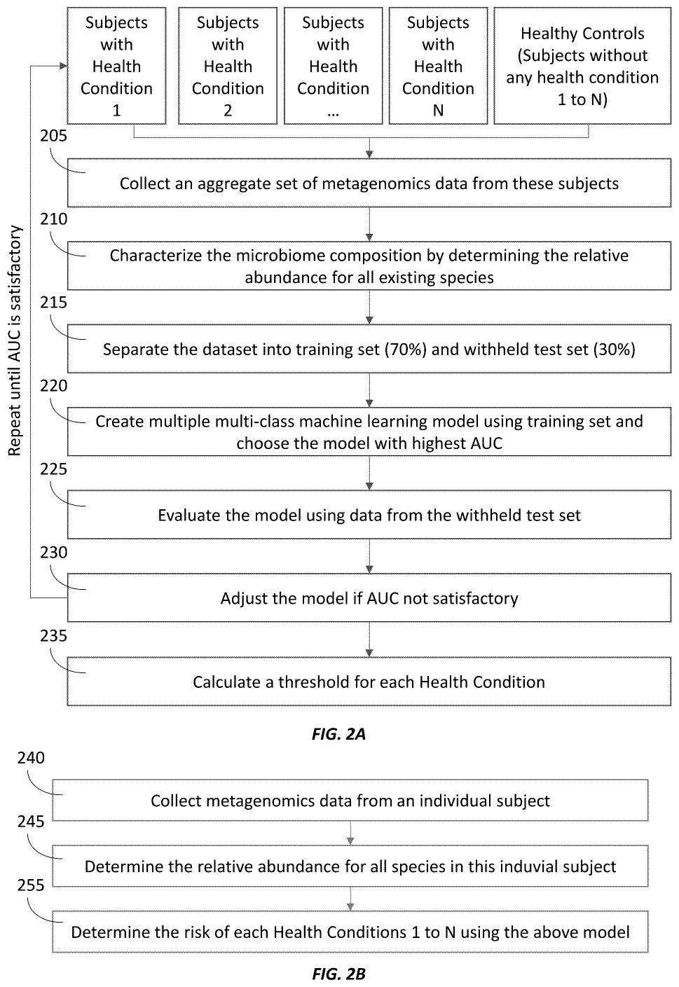

FIG. 2A is a flowchart showing an overview of generating a multi-class machine learning model according to an embodiment. In some cases, the generation of multi-class machine learning model is an ongoing process, which includes constant update, validation, and improvement. When new data is collected, the model can be updated and validated. To generate the multi-class machine learning model, the following steps can be carried out:

At block 205, an aggregate set of metagenomics data can be collected from subjects with known health conditions. The health condition phenotypes can include diseases and healthy control phenotypes.

At block 210, the microbiome composition of these subjects can be characterized by determining the relative abundance for all species existing in any one or more of the subjects. For instance, by shotgun metagenomic sequencing of fecal samples.

At block 215, separate the dataset into a training set (e.g., 70%) and a withheld test set (e.g., 30%).

At block 220, multiple multi-class machine learning models can be created from algorithms using training set data. An algorithm can be trained into a model using the cross-validation (CV) method and after training, the best model can be selected. A random forest algorithm can be trained to produce a random forest classifier. Other algorithms can be trained to create machine learning models including a multi-layer perceptron (MLP) algorithm, a support vector machine (SVM) algorithm, a K nearest neighbors (KNN) algorithm, and a Graph convolutional neural network (GCN) algorithm. For example, the CV method can be used to create 100 models with 20 times repeat and 5-fold training by dividing the training set into 8:2 ratio. 80% of the training set can be used for training, and 20% used for within-training set validation. Then, choose the model with highest AUC from these 100 models.

A k-fold hyperparameter optimization procedure can be nested inside the k-fold cross-validation for algorithm selection to further improve the model performance (e.g. evaluated by models' AUC) while reducing the problem of overfitting. In the inner loop, the models can be tuned by changing the hyperparameters (e.g. if random forest is used, adjust the hyperparameter n_estimators and, for example, the parameter can be set to n_estimators=2000).

At block 225, the models can be evaluated using data from test set to obtain the models' AUC (e.g., the probability that the model can correctly distinguish between a randomly selected correctly classified sample and an incorrectly classified sample).

At block 230, the model can be adjusted by increasing number of subjects in training set if the AUC is not satisfactory. The AUC may not be satisfactory if the value is below a threshold.

Repeat blocks 205 to 230 can be repeated until the AUC from block 225 is satisfactory and a final model can be obtained. Whether an AUC is acceptable can vary depending on the health condition(s) being tested. Lower AUC scores may indicate a higher level of confidence and a higher AUC score can indicate a lower level of confidence. An acceptable threshold can be an AUC value that is greater than or equal to 0.01, 0.05, 0.1, 0.2, 0.3, 0.4, 0.5, 0.6, 0.7, 0.8, 0.9, 1.0.

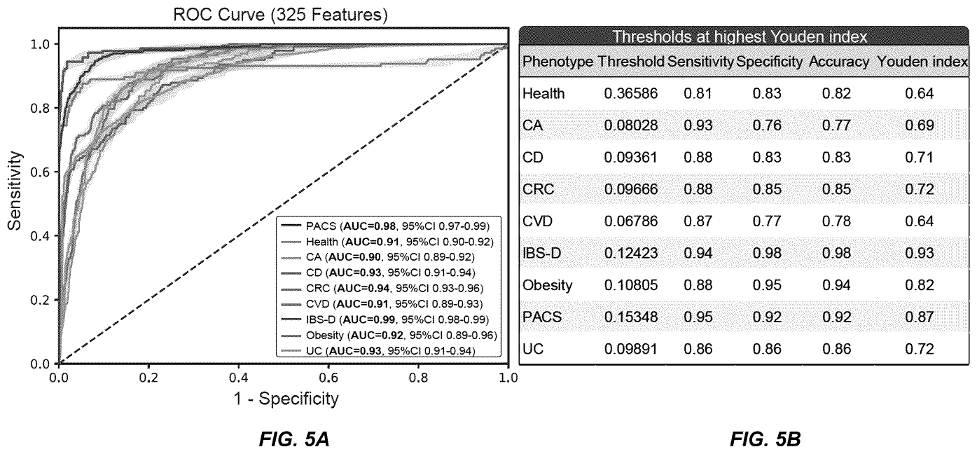

At block 235, an ad hoc threshold of the final model can be calculated for each health condition phenotype based on the Youden Index. The Youden index can integrate sensitivity and specificity information, and by using Youden index analysis, we could find that the optimal cutoff value such as the value providing the best tradeoff between sensitivity and specificity. The Youden index is discussed in greater detail in the description for FIG. 5B below.

FIG. 2B is a flowchart showing techniques for applying the model to predict risk of pre-determined health conditions in a subject according to an embodiment. To determine whether an individual is at risk of the pre-defined health conditions, the following steps can be carried out:

At block 240, collect metagenomics data from an individual subject. The metagenomics data can be determined by shotgun metagenomic sequencing of a fecal sample.

At block 245, determine the relative abundance for all species in the subject.

At block 250, if a random forest classifier is generated at block 220, decision trees will be generated by random forest from the training data. The relative abundances and values of other clinical factors will be run down the decision trees and a risk score (probability) can be generated for each pre-defined health condition. The test subject can be determined to be at risk of the pre-defined health condition if the risk score is higher than the thresholds calculated at block 235.

### II. Example Results: Fecal Microbiome-Based Machine Learning for Multi-Class Disease Diagnosis

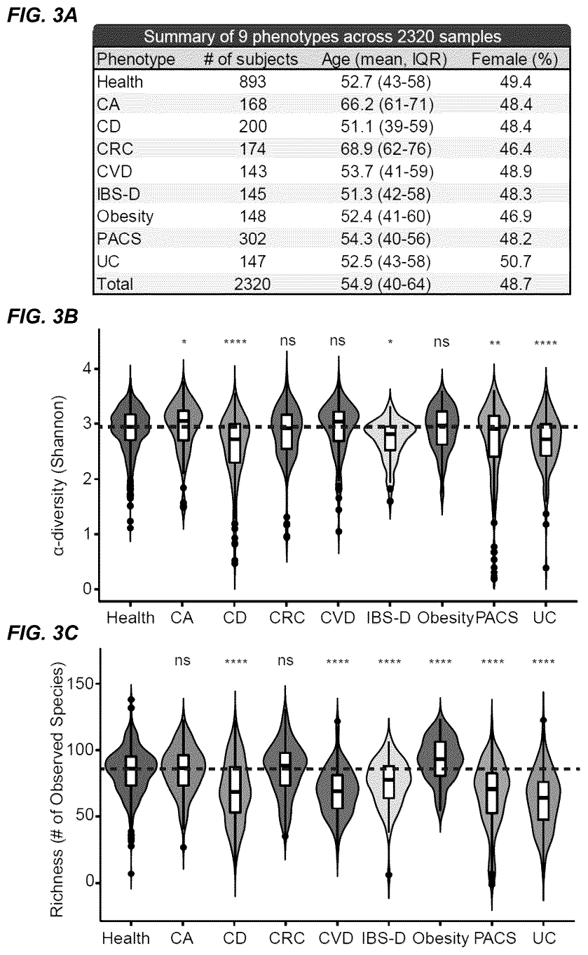

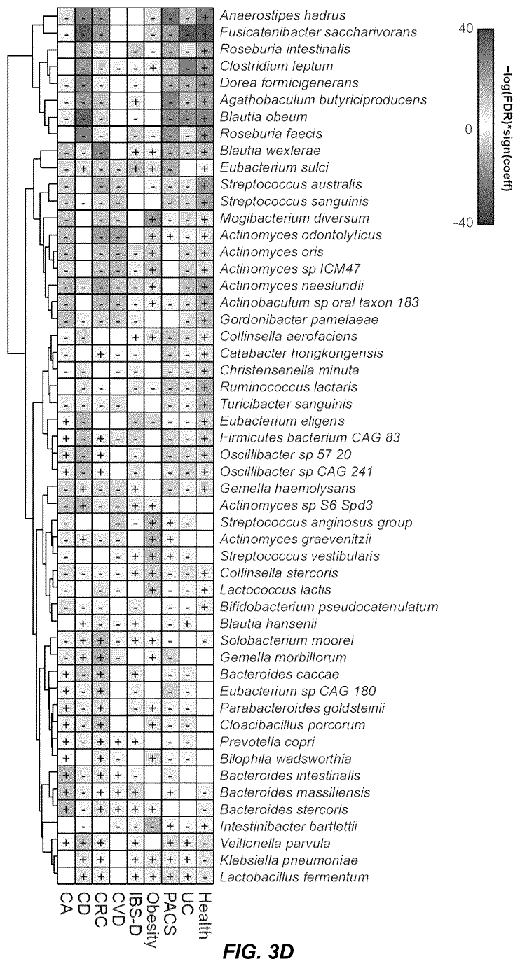

FIG. 3A is a table showing a summary of the demographics of the individuals providing fecal samples to an embodiment. We performed metagenomic sequencing at an average depth of 6.45 gigabase on fecal samples of a cohort of 2,320 Hong Kong Chinese individuals (average age 54.9, 48.7% female, FIG. 3A) consisting of nine well-characterized disease phenotypes: CRC (n=174), colorectal adenomas (CA, n=168), Crohn's disease (CD, n=200), ulcerative colitis (UC, n=147), irritable bowel syndrome (IBS, subtype D, n=145), obesity (n=148), CVD (atherosclerotic, n=143), PACS (n=302), and healthy controls (n=893). In total, we obtained 14.6 terabyte of the sequence, and 1,208 bacterial species were detected in at least one metagenome sample. Of them, 325 bacterial species had a relative abundance higher than 0.15% and these species were present in over 5% of the subjects. This sample size and sequencing depth allow us to capture the majority of microbial profile which covers over 95% of the total expected number of microbial taxa at the species level by bootstrap analysis (325 out of 339.6, Standard error=2.9). Individuals with CA or CRC are older than that of subjects in other phenotypes (p<0.05), but no difference was found in gender among different phenotypes.

FIG. 3B is a graph showing the alpha diversity of bacteria in the cohort according to an embodiment. Diversity can be the range of different kinds of species, such as unicellular organisms, bacteria, archaea, protists, and fungi, within a community. Alpha diversity can be a measure of the number of species within a community and the relative abundance of each species within the community. In this instance, the community can be an individual in the cohort and the species are bacterial species. FIG. 3A shows diversity calculated using the Shannon Index which is defined in the following equation:

$${H}^{′} = - \sum [ ( {p}_{i} ) * ln ( {p}_{i} ) ]$$

Where H′ is the Shannon Index value and pᵢ the proportion of the total population within a community represented by species i. P values were calculated by comparing the healthy controls to the other health conditions using MaAsLin 2 after adjustment of age and sex.

FIG. 3C is a graph showing the richness of fecal biome bacteria in the cohort according to an embodiment. Richness can be the observed number of species within a community. In this case, the species can be the number of bacterial species and the community can be an individual in the cohort. P values were calculated by comparing the healthy controls to the other health conditions using MaAsLin 2 after adjustment of age and sex.

Deep shotgun metagenomic sequencing provides the opportunity to explore the common set of microbial species (common core, 325 species) in our cohort. We observed different alterations in bacterial diversity (Shannon Index) and richness (observed number of bacterial species) in different disease-versus-health comparisons. For example, bacterial diversity was significantly higher in fecal samples of colorectal adenomas (p<0.05) but lower in that of CD (p<0.0001) compared with healthy controls. These results are consistent with a meta-analysis [13], suggesting that ecological indices (e.g. Shannon diversity and richness) may not be robust predictors of health or disease.

FIG. 3D is a graph showing the association of fecal biome bacterial species to the health condition phenotypes according to an embodiment. The top 50 microbial species with the highest number of associations were visualized. Associations were colored by direction of effect (red, positive; blue, negative; p<0.05), with associations significant at FDR<0.05 marked with plus (positive correlations) or minus (negative correlations), respectively. We explored the associations of microbial composition at species level to the above nine disease or healthy phenotypes using a linear model of MaAsLin2 after adjustment for age and gender. We found a total of 1,061 significant associations between these nine phenotypes and 215 bacterial taxa at the species level (FDR<0.05). Different disease phenotypes had distinct numbers of associations with different bacterial species. CD exhibited the strongest associations (defined by the total number of bacteria-phenotype associations; 143 associations), followed by PACS (140 associations) and CRC (125 associations), as compared to CVD (69 associations).

Within the identified 215 disease-related bacterial species, more than 94% (203/215) was significantly associated with two or more diseases, and about 74% (159/215) was associated with four or more diseases. For instance, Klebsiella pneumoniae, a well-characterized opportunistic pathogen [14], was positively associated with CD, CRC, IBS-D, Obesity, PACS and UC in our cohort, whilst Roseburia intestinalis, a potential probiotic with butyrate producing properties [15], negatively correlated with these six health condition phenotypes (FIG. 2D). Consistent with previous findings [12,13], our data exhibited shared microbiome associations across different diseases, suggesting that single-disease diagnostic models are likely to be confounded by unrelated diseases [12].

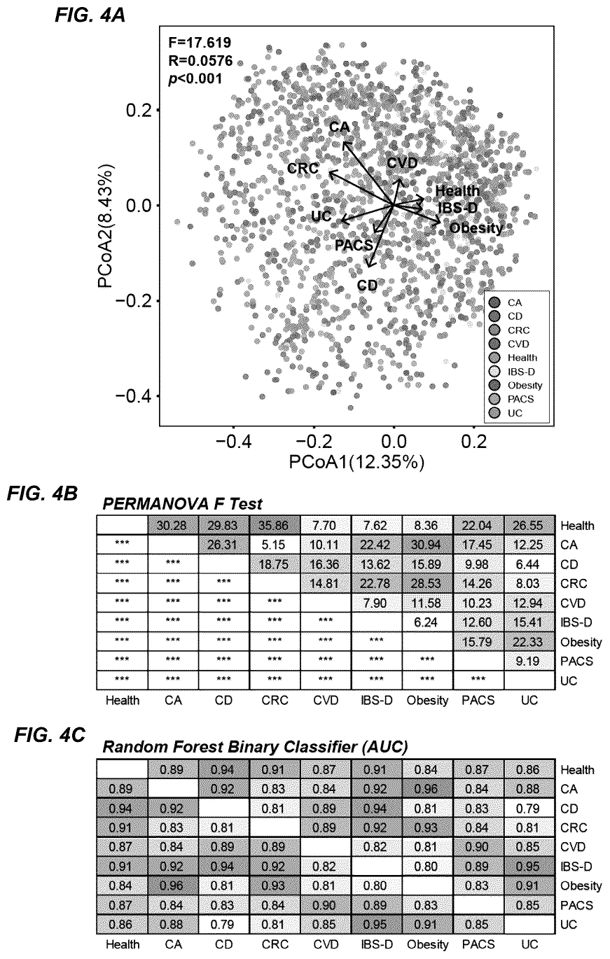

FIGS. 4A-4C show fecal microbiome differences among health conditions according to an embodiment. FIG. 4A is a graph showing a principal coordinates analysis (PCoA) of fecal biome beta diversity. Beta diversity can be a measure of the similarity, or dissimilarity, of different communities. The beta diversity can be a ratio between the diversity of an individual in a cohort and the total diversity of the cohort. PcoA is a type of multidimensional scaling that creates mappings of entities based on distance. FIG. 4B is a chart showing the results of a permutational analysis of variance (PERMANOVA) test of fecal biome beta diversity for one health condition versus another. PERMANOVA is a nonparametric alternative to multivariate analysis of variance (MANOVA), and PERMANOVA compares groups of entities against a null hypothesis that there are no differences between the entities.

FIG. 4C is a chart showing area under the receiver operating characteristic curve (AUC) of random forest (RF) binary classifiers for one versus one discrimination of multiple health conditions. We measured microbiome compositional differences between different phenotypes both by beta-diversity-based permutation tests and by machine-learning binary classifications (random forest, RF). Our data showed that PcoA analysis based on beta-diversity separated the different disease phenotypes (FIG. 4A-4B), and these differences of microbiome composition between different phenotypes were also reflected in the area under the receiver operating characteristic curve (AUC) of random forest (RF) binary classifiers (one versus one, average 0.87, interquartile range (IQR) 0.83-0.91, FIG. 4C). Taken together, although there are numerous shared microbial markers across diseases, these findings demonstrated that microbial compositions still vary by disease states.

FIG. 5A shows a graph of a receiver operating characteristic (ROC) curve for the random forest (RF) multi-class classifier according to an embodiment. The figure also lists the AUC for the nine health conditions evaluated by the RF multi-class classifier using data from sequenced fecal samples. Our RF multi-class classifier achieved a mean AUC of 0.90 to 0.99 (Interquartile range, IQR 0.91-0.94, one versus all others) for different disease phenotypes.

FIG. 5B shows a chart of thresholds at the highest Youden index for the random forest (RF) multi-class classifier according to an embodiment. The Youden index is a statistic that combines specificity and sensitivity in a single value. The Youden index can be calculated with the following formula:

$$Y = sensitivity + specificity - 1$$

The specificities for the classifier shown in FIG. 5B ranged from 0.758 to 0.982 (IQR 0.83-0.95, one versus all others) for different health conditions, highlighting good diagnostic performance using this multi-class classifier. For example, our classifier achieved a mean AUC of 0.94 for CRC with a sensitivity of 0.877 and specificity of 0.846 (one versus all others, FIG. 5A-B); this performance exceeded that of our own trained binary classifier (CRC versus health, mean AUC 0.91, FIG. 4C) and a previously published CRC diagnostic model [4]. One vs all others can mean that a classifier is trained for each health condition and the output of a health condition's classifier is the probability that the sample is from someone with the health condition or it is a sample from someone with any of the other health conditions.

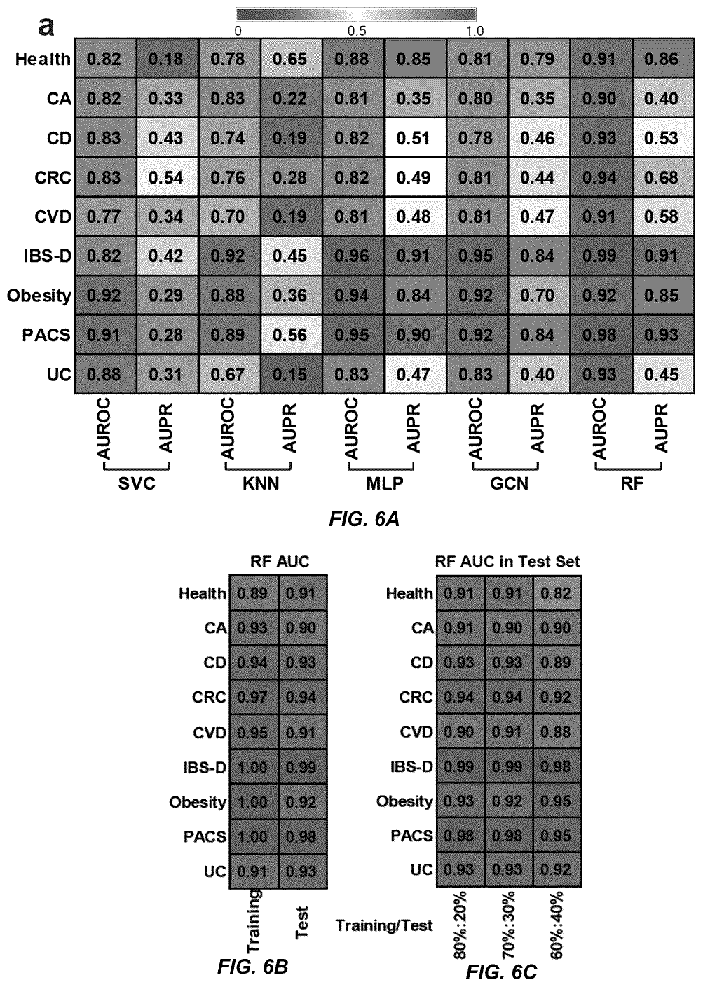

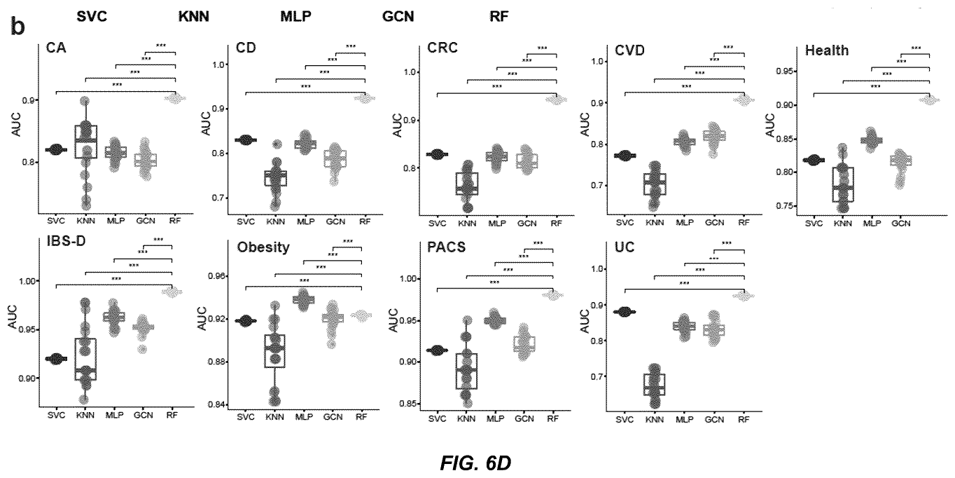

FIG. 6A-6D show charts with the AUC and AUPR for models trained to classify health conditions according to an embodiment. The figure lists nine health conditions and four models including a support vector multi-class classifier (SVC), a k nearest neighbor (KNN) multi-class classifier, a random forest (RF) multi-class classifier, a multilayer perceptron (MLP) multi-class classifier, and a Graph convolutional neural network (GCN) multi-class classifier. The RF multi-class classifier performed well with a mean area under the precision-recall curve (AUPR) of 0.40 to 0.93 (IQR 0.53-0.86, FIG. 6) which generally outperformed all other models (FIG. 6). At a threshold based on the highest Youden's Index, the sensitivities of our RF multi-class classifier ranged from 0.807 to 0.946 (IQR 0.87-0.93).

To fully characterize the RF multi-class model, we compared its performance under different split ratios and achieved similar results, suggesting high stability and good predictive power without risk of overfitting (FIG. 6C). These models achieved a mean AUROC (e.g., AUC) of 0.67 to 0.99 (Interquartile range, IQR 0.81-0.92), suggesting that multi-class disease classification based on the faecal microbiome was feasible. Amongst them, the RF multi-class model achieved a mean AUROC of 0.90 to 0.99 (IQR 0.91-0.94, one versus all others) for different disease phenotypes in the test set. The performance of the RF model in the test set generally outperformed all other models (FIG. 6D) and was similar to that of the training set (calculated by 5-fold cross-validation, FIG. 6B), suggesting high integrity of this classifier.

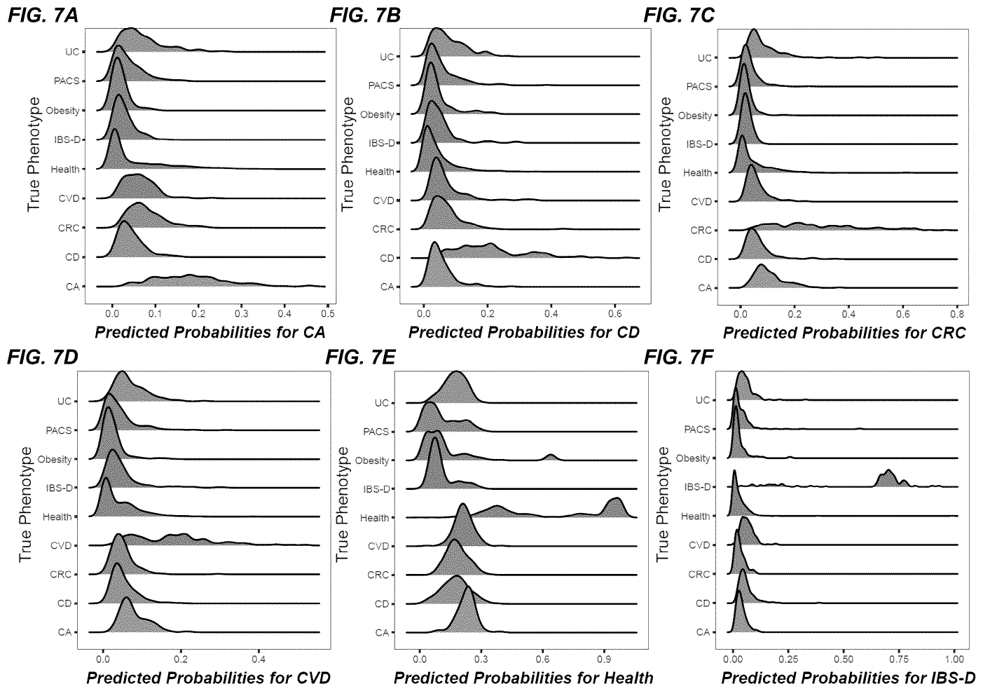

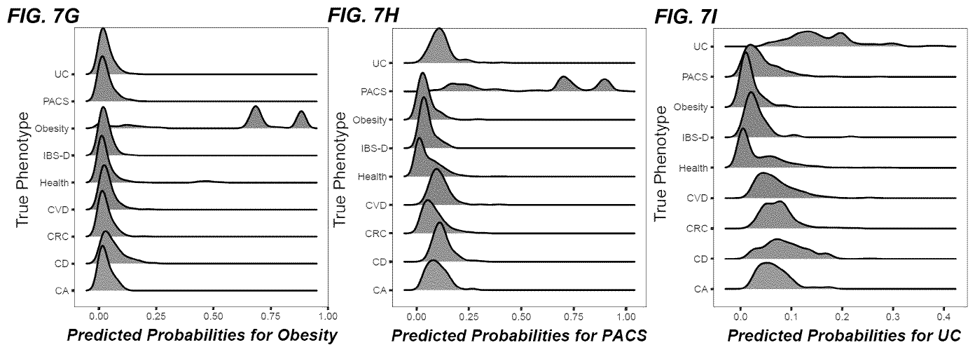

FIG. 7A-7I shows graphs of the distribution of probabilities yielded by the trained random forest (RF) multi-class classifier according to an embodiment. The probabilities were calculated for multiple health conditions during independent test. In independent test, the multi-class classifier evaluated a dataset containing a single health condition. The probabilities were calculated for the health conditions in the test set. FIG. 7A shows probabilities for colorectal adenomas (CA); 7B shows probabilities for Crohn's disease (CD); 7C shows probabilities for colorectal cancer (CRC); 7D shows probabilities for Cardiovascular disease (CVD); 7E shows probabilities for a healthy condition; 7F shows probabilities for diarrhea-dominant irritable bowel syndrome (IBS-D); 7G shows probabilities for Obesity; 7H shows probabilities for post-acute COVID-19 syndrome (PACS); and 7I shows probabilities for ulcerative colitis (UC).

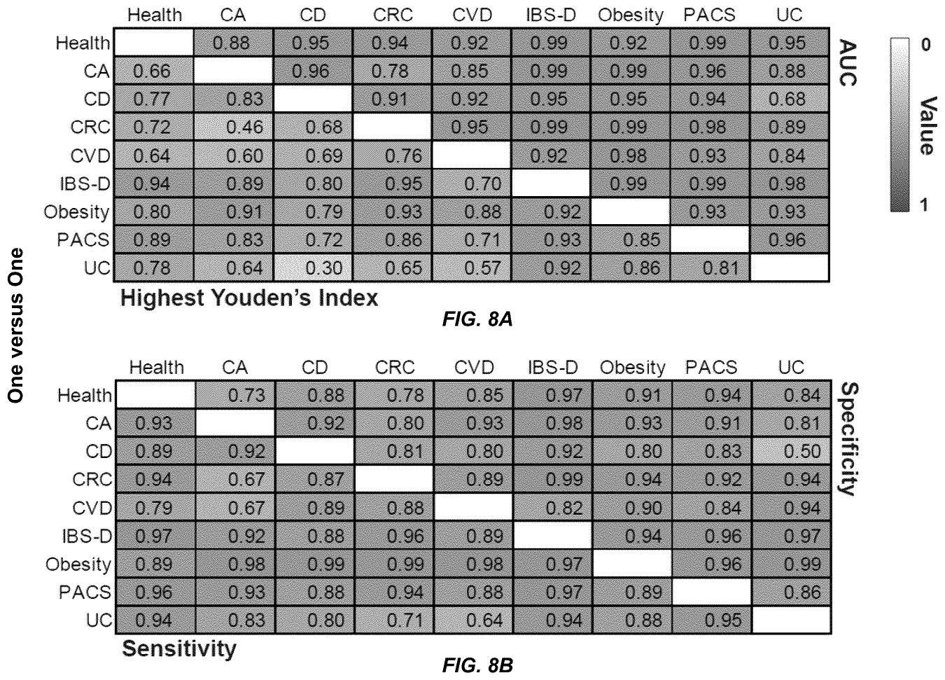

FIGS. 8A-8B show the performance of the random forest (RF) multi-class classifier in one versus one discrimination of multiple health conditions according to an embodiment. FIG. 8A shows area under the receiver operating characteristic curve (AUC) and the highest Youden's index of the random forest multi-class classifier using species-level fecal microbiome data. FIG. 8B shows sensitivities and specificities selected based on the highest Youden's index for one versus one discrimination of multiple phenotypes. The performance was evaluated using predicted probabilities in the test set (FIGS. 7A-7I) and further assessment showed that the trained classifier achieved a mean AUC of 0.94 for all one-versus-one classification (IQR 0.92-0.98, FIG. 8A) with high sensitivities (IQR 0.88-0.95) and specificities (IQR 0.83-0.94, FIG. 8B), which supported a superior performance of multi-class model analyses over binary models (FIG. 5B).

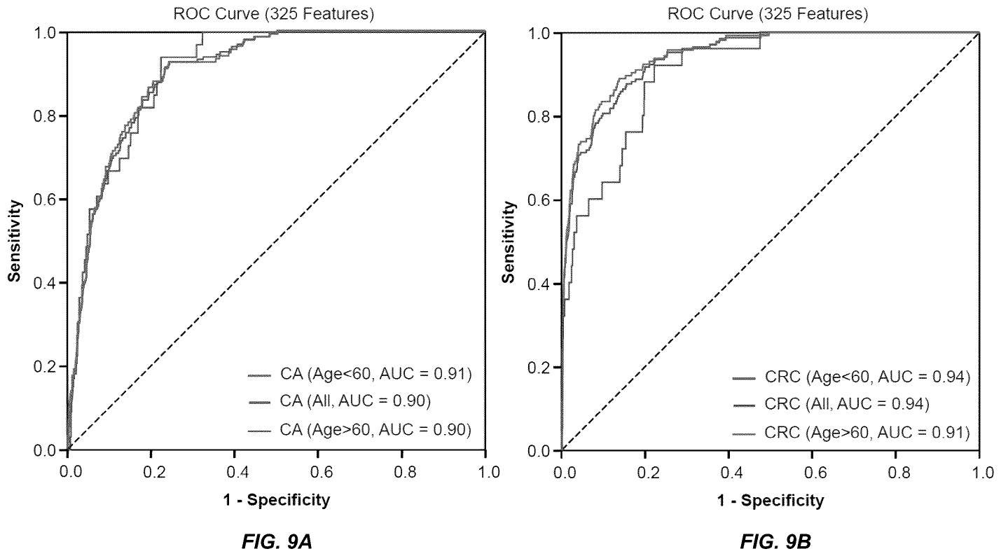

FIGS. 9A-9B are graphs showing the performance of random forest (RF) multi-class classifier stratified by age. FIG. 9A shows the performance of the RF multi-class classifier for colorectal adenomas (CA) and FIG. 9B shows the performance of the classifier for colorectal cancer. Given that age of subjects with CRC or CA were older (FIG. 3A), we assessed the performance of our model stratified by age and found consistent results.

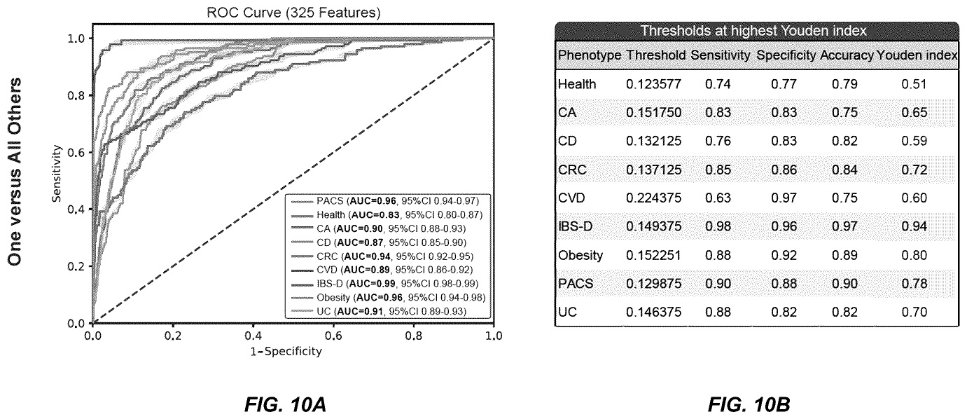

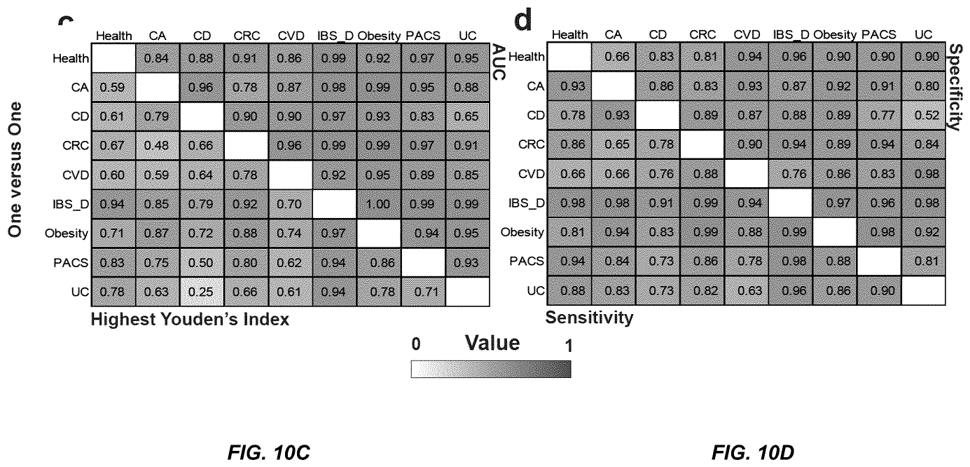

FIGS. 10A-10D show the performance of a random forest (RF) multi-class classifier that was trained and tested on a balanced cohort size according to an embodiment. FIG. 10A shows the area under the receiver operating characteristic curve (AUC). FIG. 10B shows model performance metrics details of random forest multi-class classifier for diagnosing one phenotype versus all others using species-level fecal microbiome data. FIG. 10C shows the AUC and the highest Youden's index of the random forest multi-class classifier for one versus one discrimination of multiple phenotypes.

FIG. 10D shows the sensitivities and specificities for the classifier that were selected based on the highest Youden's index for one versus one discrimination of multiple phenotypes. To examine whether uneven cohort sizes across the nine phenotypes influenced classification performance, we trained a RF multi-class classifier by randomly pooling 143 subjects from each disease phenotype (a total of 1,287 subjects, 70% training, 30% testing) following the same process (FIG. 5A). We observed that this classifier performed (AUC IQR 0.91-0.94, one versus all others; IQR 0.89-0.96, one versus one; FIGS. 10A-10D) comparable to the non-subset cohort of 2,320 individuals (AUC IQR 0.89-0.96, one versus all others; IQR 0.92-0.98; FIG. 5B, FIGS. 8A-8B), indicating that the unbalanced sample size has negligible influence on the performance.

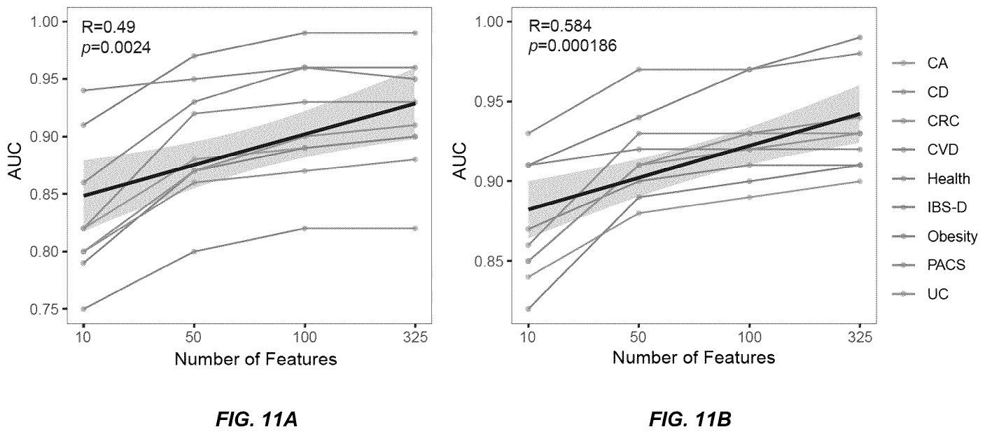

FIG. 11A-11B show the performance of a random forest (RF) multi-class classifier using different numbers of features. FIG. 11A shows the area under the receiver operating characteristic curve (AUC) of random forest multi-class classifier for diagnosing one phenotype versus all others using top 10, 50, 100 and all 325 features in the balanced sample size cohort (n=1287). FIG. 11B shows the area under the receiver operating characteristic curve (AUC) of random forest multi-class classifier for diagnosing one phenotype versus all others using top 10, 50, 100 and all 325 features in the complete cohort (n=2320). The AUC values of the model increased with increasing number of features which excluded the possibility of overfitting based on the 325 features (FIGS. 11A-11B).

FIGS. 12A-12C show independent validations of the performance of the random forest (RF) multi-class classifier according to an embodiment. 12A shows a summary of the health conditions, sample size, and the source countries for the publicly available cross-regional datasets used in the independent validations. 12B shows the area under the receiver operating characteristic curve (AUC) of the trained RF multi-class classifier for diagnosing one phenotype versus all others in the cross-regional datasets. 12C shows the probabilities yielded by the trained random forest multi-class classifier for 60 subjects who post COVID-19 but did not have post-acute COVID-19 syndrome. The independent validations were determined by integrating 1,597 shotgun stool metagenome data from 12 published studies from 11 countries, covering Asia, Europe and North America (FIG. 12A). The RF multi-class classifier showed a mean AUC of 0.82, IQR 0.79-0.87, FIG. 12B) for classifying different diseases.

Specifically, the AUC for diagnosis of CRC, IBS-D and CVD were 0.82, 0.86 and 0.88, respectively. Such performance from independent cross-regional validation further confirmed the robustness and generalizability of our model across populations and geography. Besides, to further validate the accuracy of the RF model, we selected 60 patients who had complete recovery from COVID-19, as it has been shown that the gut microbiome of COVID-19 survivors with no long-term symptoms were similar to that of controls [10]. We found that the trained model had a specificity of 83.3% (50/60) in diagnosing these subjects as healthy (FIG. 12C) which verified that fully recovered COVID-19 survivors had similar gut microbiome profiles as healthy people.

We integrated 1,597 shotgun fecal metagenome data from 12 public datasets from Asia, Europe and North America (FIG. 12A). Our RF multi-class classifier showed a mean AUROC of 0.69 to 0.91 (IQR 0.79-0.87) in classifying different diseases (FIG. 12B). Such performance from an independent validation cohort indicated the robustness and generalizability of the model across different populations and geographical locations. To further validate the accuracy of the model, we selected 60 patients who had a complete recovery from COVID-19 infection. Our trained model showed an accuracy of 83.3% (50/60) in classifying these subjects as healthy (FIG. 12C). These data verified that fully recovered COVID-19 survivors (and without PACS) shared similar gut microbiome profiles as healthy people. Additionally, we also tested our RF model on unrelated liver cirrhosis and constipation-dominant IBS datasets (n=60). The results showed that 12 subjects are predicted as Health (n=1), CD (n=3), CRC (n=3), PACS (n=3) and UC (n=2), while no prediction was made for other 48 subjects because the highest predicted probabilities for them fail to exceed corresponding thresholds (FIG. 12D). These results suggested the high specificity and accuracy of our model, which has a low chance of misclassifying unrelated diseases into nine phenotypes analyzed in this study.

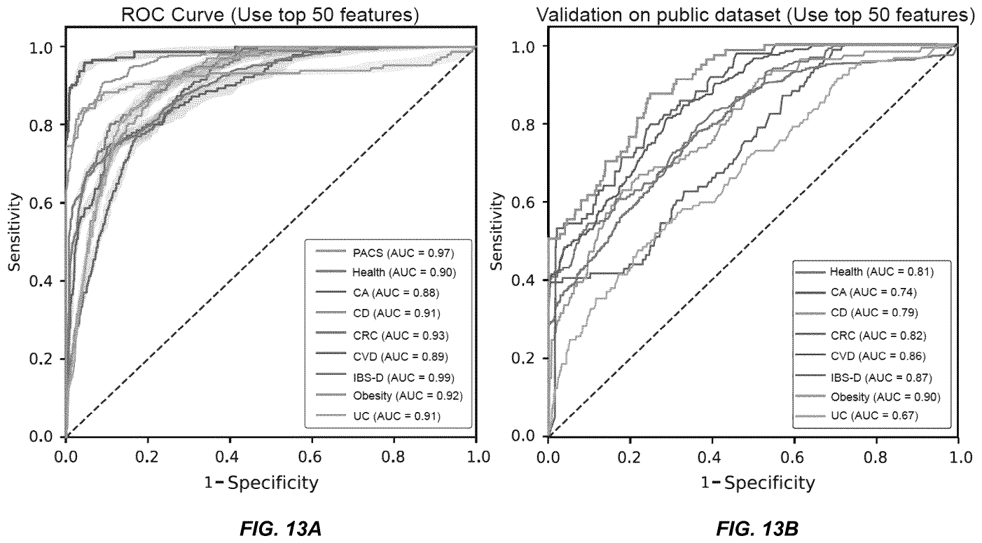

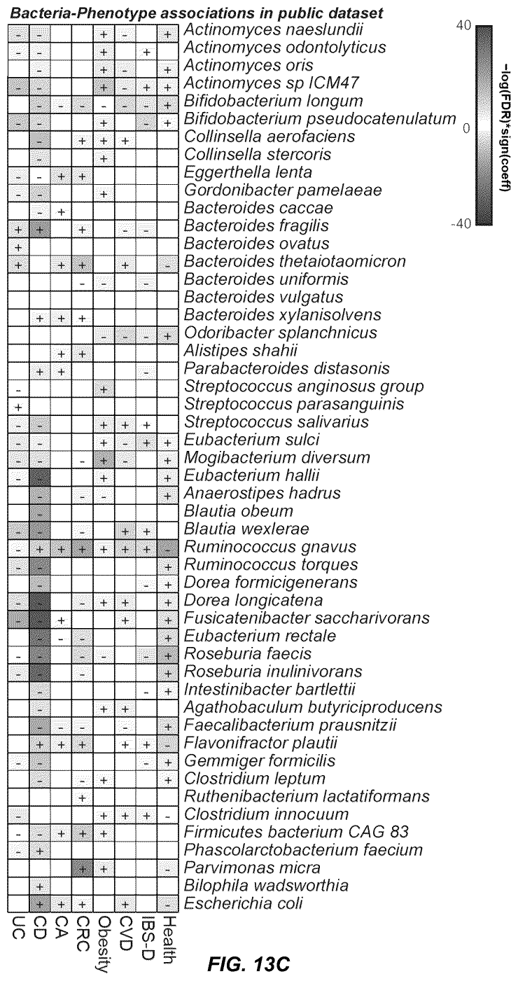

FIG. 13A-13C show the performance of the random forest (RF) multi-class classifier using the top 50 features according to an embodiment. FIG. 13A shows the area under the receiver operating characteristic curve (AUC) of the RF multi-class classifier (using top 50 features) for diagnosing one phenotype versus all others in the independent test set. FIG. 13B shows validation of the performance of the RF multi-class classifier (using top 50 features) in the publicly available datasets. Given the strong discrimination of microbial signatures employed in the multi-class classifier, we correlated top model contributors (top 50 bacterial species contributing to the model, sum of importance: 0.41) and different phenotypes. These top 50 bacterial species achieved about 98.6% performance (compare the average AUC value for different phenotypes, FIG. 13A) of the complete model using 325 features. Compared with healthy controls, almost all disease states were associated with a significant decreased abundance of microbiota from the phylum of Firmicutes or Actinobacteria (FDR<0.05) and a significant increase in Bacteroidetes (FDR<0.05).

The top 50 bacterial species contributing to the model were correlated with different disease phenotypes to identify clues to model interpretability. These top 50 bacterial species achieved a mean AUROC of 0.88 to 0.99 (IQR 0.90-0.93, FIG. 13A) for different diseases in our test set, and a mean AUROC of 0.67 to 0.90 (IQR 0.78-0.86, FIG. 13B) in the public dataset. A total of 363 significant associations were found between these 50 species with different disease phenotypes. Compared with healthy controls, almost all disease states were associated with a decreased abundance of microbiota from the bacteria phylum of Firmicutes or Actinobacteria (FDR<0.05) and an increase in Bacteroidetes (FDR<0.05). Imbalance in Firmicutes/Bacteroidetes ratio had previously been reported primarily in patients with obesity and IBD, but its associations with other diseases have not been reported. Nonetheless, such shared microbial signatures may serve as a basis for distinguishing health and disease. Then, we identified microbial signatures that can classify different diseases. Specifically, the abundance of several bacterial species in Bacteroidetes differed significantly between patients with PACS, UC and CD. Subjects with PACS showed a significant increase in abundance of Bacteroides vulgatus and Bacteroides xylanisolvens, while those with UC were enriched in Bacteroides ovatus, and subjects with CD showed significant decreases in Bacteroides uniformis, Bacteroides vulgatus and Bacteroides xylanisolvens, compared with healthy controls.

Although patients with CRC and colorectal adenomas shared relatively similar gut bacteria composition, the abundance of Parvimonas micra was significantly higher in patients with CRC but not colorectal adenomas, compared to healthy controls, which was consistent with previous findings showing that Parvimonas micra can be used as a marker to distinguish CRC from colorectal adenomas. For other diseases, microbiome differences were mainly driven by Actinobacteria. Subjects with obesity showed increases in Actinomyces naeslundii, Actinomyces odontolyticus and Actinomyces oris, and subjects with IBS-D showed increases in Collinsella aerofaciens and Collinsella stercoris. We further correlated bacteria and phenotypes in the assembled public dataset and found that many disease-specific biomarker are stable across datasets, such as Bacteroides for UC, Parvimonas micra for CRC and Actinomyces for obesity (FIG. 13C). Overall, these results suggest that our model can capture various disease-specific microbial signatures, which may explain the robust diagnostic performance of this multi-class classifier.

Overall, our data showed that the fecal microbiome-based multi-class model for disease diagnosis is feasible. The novelty lies in the high-quality dataset, superior and reproducible machine-learning methods which is of high clinical relevance, and our classifier can be used as a rapid screening method for various diseases in daily medical examination or disease risk assessment. Our results also have implications for the potential development of biomarkers for predicting drug response and common treatment strategies using the identified shared or specific marker for multiple diseases. This work has some limitations. Since our model predicts probabilities for multiple diseases simultaneously, it may also apply to multi-disease diagnosis in a single patient.

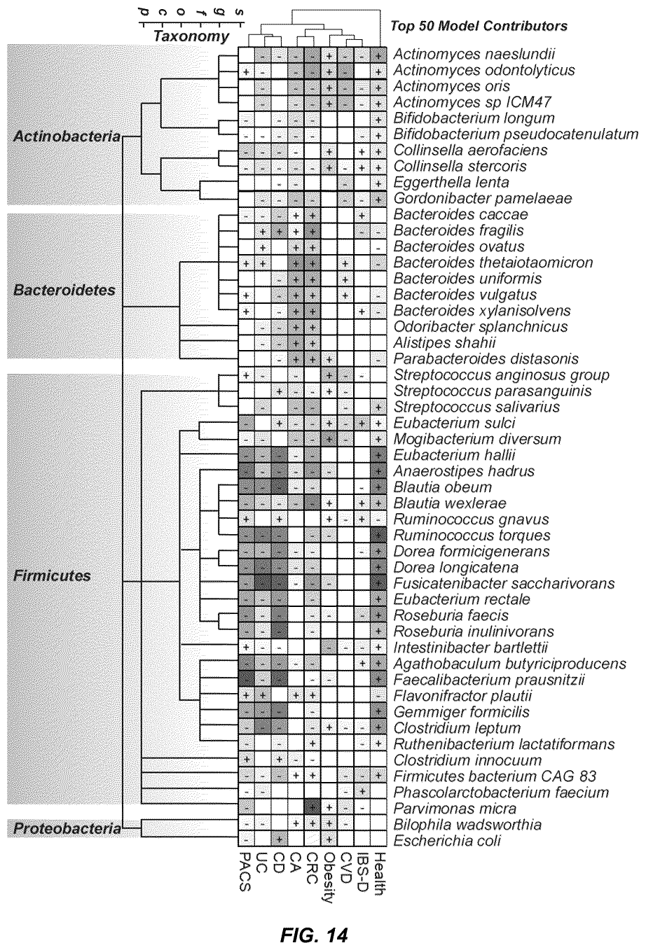

FIG. 14 is a chart chowing microbial species associated with health status or different health conditions according to an embodiment. The top 50 microbial species with the highest contribution to the random forest multi-class classifier were clustered by taxonomy, and different phenotypes were clustered using hierarchical clustering. Associations were colored by direction of effect (red, positive; blue, negative; p<0.05), with associations significant at FDR<0.05 marked with plus (positive correlations) or minus (negative correlations), respectively. CA, colorectal adenomas; CD, Crohn's disease; CRC, colorectal cancer; CVD, Cardiovascular disease; IBS-D, diarrhea-dominant irritable bowel syndrome; PACS, post-acute COVID-19 syndrome; UC, ulcerative colitis.

Imbalance in Firmicutes/Bacteroidetes ratio has previously been reported primarily in patients with obesity and IBD [17], but its associations with other diseases remains limited. Nonetheless, these shared microbial signatures may serve as a basis for distinguishing health and disease (FIG. 14). Furthermore, we observed the microbial signatures that can classify different disease phenotypes (FIG. 14). The abundance of several bacterial species in Bacteroidete differed significantly between patients with PACS, UC and CD. Subjects with PACS showed a significant increase in Bacteroides vulgatus and Bacteroides xylanisolvens, while those with UC were enriched in Bacteroides ovatus, and subjects with CD showed significant decreases in Bacteroides uniformis, Bacteroides vulgatus and Bacteroides xylanisolvens, compared with healthy controls. Although the gut microbiome of patients with CRC and colorectal adenomas shared relatively similar microbiome taxa, we found that abundance of Parvimonas micra was significantly higher in patients with CRC but not in colorectal adenomas, when compared to healthy control, which was consistent with previous findings showing that Parvimonas micra can be used as a marker to distinguish CRC from colorectal adenomas [18,19]. As for other diseases, differences were mainly from Actinobacteria. Subjects with obesity showed increases of Actinomyces naeslundii, Actinomyces odontolyticus and Actinomyces oris, and those with IBS-D had a specific increase in Bifidobacterium pseudocatenulatum. Overall, these results suggested that our model captured various disease-specific microbial signatures, which may explain the good diagnostic performance of this multi-class classifier.

Shared microbial signatures across different disease phenotypes is one of the largest challenges in developing microbiome-based biomarkers for disease diagnosis [12]. Our work confirmed that fecal microbiome-based multi-class disease diagnosis is feasible, and some microbial taxa are unique to specific disease phenotype. Machine learning model using multi-class approaches could achieve a higher performance than traditional single-disease models, probably because it can better focus on disease-specific signals and is not sensitive to overfitting. All these results revealed that our fecal microbiome-based machine learning for multi-class disease diagnosis can be applied more broadly.

Our results also have implications for the development of potential treatment options for multiple diseases by providing potential microbial targets that are of diagnostic and therapeutic value. A list of bacteria that can be used to treat diseases are included in table 4. For example, we observed the negative associations between Roseburia intestinalis and six diseases, thus this species may provide a range of health benefits and have the potential to be developed as a general-purpose probiotic. However, the limitation was that there is little biological evidence related to our identified microbiome-phenotype associations. Future works explaining the causality of these associations can help understand the role of gut microbiome in diseases pathogenesis. As for other limitations, the pooled public dataset may have various confounding factors such as comorbidities and antibiotics, thus the validation performance may vary with proper participants exclusion.

Overall, we present the largest fecal microbiome dataset from mixed diseases cohort to date, and we trained a machine learning multi-class classifier that achieves accurate diseases classification. This fecal microbiome-based non-invasive diagnostic tool could be applicable to current clinical practice, as combination of different methods tend to further improve the efficiency of diseases screening. For example, the combination of fecal microbiome test and immunochemical test exhibited higher sensitivity for CRC [4]. Therefore, this new class of non-invasive diagnostic tool may provide substantial future value to the public.

### III. Example Implementation Details

### Study Population

All participants were recruited and diagnosed at the Prince of Wales Hospital in Hong Kong from January 2017 to March 2022. Subjects with CRC and CA were diagnosed by colonoscopy and confirmed on histology examinations; Subjects with CD and UC were diagnosed based on standard criteria of endoscopy, radiology, and histological examinations. Subjects with IBS were diagnosed according to the ROME III criteria, and endoscopy and enteroscopy were performed to exclude other GI disorders such as IBD, cocliac disease, parasite infestations, or other organic disorders. Obesity was defined as subjects with a body mass index (BMI) of over 28 and with no other medical co-morbidities. Subjects with cardiovascular disease (CVD) were recruited from the public as part of a survey of cardiovascular health in the Hong Kong general population. Subjects underwent carotid ultrasounds to measure intima-media thickness (IMT) of the common, internal, external carotid arteries (CCA, ICA and ECA, respectively) and carotid bulbs and subjects that had ≥50% stenosis in a single or multiple vessels were regarded as having the risk of CVD. Subjects with the post-acute covid-19 syndrome (PACS) were defined as those with at least one persistent symptom or long-term complications of SARS-COV-2 infection beyond 4 weeks from the viral clearance which could not be explained by an alternative diagnosis, and we assessed the presence of the 30 most commonly reported symptoms post-COVID after illness onset [10,20]. All subjects with other diseases (apart from the obesity group) had a normal range of BMI of 18.5 to 22.9. All subjects are on a stable traditional Chinese style diet and are of Han Chinese ethnicity. Patients were excluded if they had the following: age under 18 or over 80; self-reported comorbidities of other diseases; infection with an enteric pathogen; acquired immunodeficiency syndrome; known history of organ dysfunction or failure and abdominal surgery; active malignancy or undergoing radio-chemotherapy; short bowel syndrome; taking drugs commonly known to affect the gut microbiome including proton pump inhibitors, oral anti-diabetics, non-steroidal anti-inflammatory drugs, corticosteroids, laxatives or selective serotonin reactive inhibitors and antibiotics or probiotics use within three months of sample collection; pregnant or breastfeeding; on special diets such as vegetarians.

Healthy controls were recruited during the same recruitment period from the community through advertisement and from the endoscopy center at the Prince of Wales Hospital in subjects who had a normal colonoscopy (stools collected before bowel preparation). The exclusion criteria for healthy controls were known complex infections or sepsis; known history of severe organ failure (including decompensated cirrhosis, malignant disease, kidney failure, epilepsy, active serious infection, acquired immunodeficiency syndrome); bowel surgery in the last 6 months (excluding colonoscopy/procedure related to perianal disease); the presence of an ileostomy/stoma; and current pregnancy; any long term drugs for chronic diseases; the use of antibiotics in the last 3 months; the use of laxatives or anti-diarrheal drugs in the last 3 months or recent dietary changes (e.g., becoming vegetarian/vegan). Finally, a total of 2,320 subjects were recruited. Clinical metadata and dietary data were collected during clinical interviews.

Besides, additional 60 subjects (mean age 53.5, 48.3% female) were prospectively followed up for up to two years after the COVID-19 infection and were confirmed to have fully recovered from the initial infection without any symptoms of PACS. These subjects serve as an independent validation cohort and provided stool samples after SARS-COV-2 clearance. All subjects were older than 18 and provided written informed consent.

### Stool Samples

Human fecal samples were collected, frozen immediately and DNA was purified by standard method. Stool samples from participants were collected by hospital staff after completing the diagnosis and recruitment procedure. All samples were collected in tubes containing preservative media (cat. 63700, Norgen Biotek Corp, Ontario Canada) and stored immediately at −80° C. until processing. We have previously shown that data on gut microbiota composition generated from stools collected using this preservative medium is comparable to data obtained from samples that are immediately stored at −80° C. [21].

### Stool DNA Extraction and Sequencing

After removing the preservative media, the fecal pellet (100 mg) was added to 1 mL of CTAB buffer and vortexed for 30 seconds, then the sample was heated at 95° C. for 5 minutes. After that, the samples were vortexed thoroughly with beads at maximum speed for 15 minutes. Then, 40 μL of proteinase K and 20 μL of RNase A were added to the sample and the mixture was incubated at 70° C. for 10 minutes. The supernatant was then obtained by centrifuging at 13,000 g for 5 minutes and was added to the Maxwell RSC machine for DNA extraction according to the instruction (Cat: AS1700, Maxwell, USA). After the quality control procedures by Qubit 2.0, agarose gel electrophoresis, and Agilent 2100, extracted DNA was subject to DNA libraries construction, completed through the processes of end repairing, adding A to tails, purification and PCR amplification, using Nextera DNA Flex Library Preparation kit (Illumina, San Diego, CA). Libraries were subsequently sequenced on our in-house sequencer Illumina NextSeq 550 (150 base pairs paired-end) at the Center for Microbiota Research, The Chinese University of Hong Kong. All samples were in random order for DNA extraction, library construction and sequencing. ZymoBIOMICS Spike-in Control I (High Microbial Load, Cat: D6320-10, ZYMO Research, USA) and ZymoBIOMICS Microbial Community DNA Standard (Cat: D6306-A) were used as positive controls during DNA extraction, library construction, sequencing and quality assessment.

### Microbiome Profiling

Raw sequence data were quality filtered using Trimmomatic V.39 to remove the adaptor, low-quality sequences (quality score <20), and reads shorter than 50 base pairs. Contaminating human reads were filtered using Kneaddata (V.0.7.2, Genome Reference database: GRCh38 p12) with default parameters. Following this, microbiota composition profiles were inferred from quality-filtered forward reads using MetaPhlAn3 version 3.0.5. GNU parallel was used for parallel analysis jobs to accelerate data processing. Alpha diversity metrics (Shannon diversity, Chao1 richness) were calculated by using the phyloseq package, version 1.26.0. Species whose average abundance and prevalence were less than 0.15% and 5% were filtered out.

### Random Forest Binary Classifier

Binary sub-cohorts were composed of two phenotypes drawn from the entire cohort. A total of 36 binary sub-cohorts were generated. Machine learning binary classifier used random forest through the Sklearn library under Python 3.6.7, as this algorithm has been shown to outperform, on average, other learning tools for microbiota data [23]. Normalized abundance table from each binary sub-cohort to train the model. Machine learning models were first trained on the randomly selected training set (70%, 5-fold cross-validation) and then were applied to the withheld validation set (30%) to access the final performance. This process was repeated 20 times to obtain a distribution of random forest prediction evaluations on the validation set, and the mean AUC value was calculated accordingly for visualization of results.

### Machine Learning for Diagnosis of Multi-Diseases

All classifier models are implemented using Python 3.6.7 using standard libraries that are publicly available: pandas (0.23.4), numpy (1.14.5), scikit-learn (1.1), and matplotlib (2.2.3). They are trained and evaluated on the microbiome datasets using the cross-validation (CV) method. The machine learning-based classifiers are implemented using the python library Sklearn [22]. For each phenotype, samples were randomly divided into a training set (70% of samples, n=1,724) and a test set for independent evaluation (remaining 30%, n=696). Random forests (RF), K-nearest neighbors (KNN), SVM multi-layer perceptron (MLP), support vector machine (SVM), and Graph convolutional neural network (GCN) were used as classifier models for the diagnosis of different phenotypes by using taxonomic profiles of the fecal microbiome. We implemented the RF multi-class classifier with the following modifications to the default SciKit-learn settings: n_estimaters=2000 and class_weight=balanced. KNN, SVM and MLP were implemented from SciKit-learn with the default settings. The optimal models selected based on cross-validated results were evaluated in the withheld evaluation dataset as the final performance for predicting incident disease. The highly ranked and frequently selected microbial features were considered predictive signatures for further interpretation. We retrieved prediction performance using the same training datasets.

### Cross-Validation

A nested cross-validation procedure was applied to calculate within-cohort accuracy by splitting data into training and test sets for 20-times repeated, fivefold-stratified cross-validation (balancing class proportions across folds). For each split, an L1-regularized (Lasso) logistic regression model was trained on the training set, which was then used to predict the test set. The lambda parameter was selected for each model to maximize the AUC-ROC under the constraint that the model contained at least five nonzero coefficients.

Binary sub-cohorts were composed of two phenotypes drawn from the entire cohort. A total of 36 binary sub-cohorts were generated. Machine learning binary classifier used random forest through the Sklearn library under Python 3.6.7, as this algorithm has been shown to outperform, on average, other learning tools for microbiota data [23]. Normalized abundance table from each binary sub-cohort to train the model. Machine learning models were first trained on the randomly selected training set (70%, 5-fold cross-validation) and then were applied to the withheld validation set (30%) to access the final performance. This process was repeated 20 times to obtain a distribution of random forest prediction evaluations on the validation set, and the mean AUC value was calculated accordingly for visualization of results.

### Statistical Analysis

Statistical analyses were done using R version 3.4.3. The ggpubr package (https://github.com/kassambara/ggpubr) performed nonparametric statistical testing between groups and accounted for multiple hypothesis testing corrections when necessary. Principal Coordinates Analysis (PCoA) was used to visualize the clustering of samples based on their species-level compositional profiles. Associations of specific microbial species with phenotypes were identified using the multivariate analysis by linear models (MaAsLin2) statistical frameworks implemented in the Huttenhower Lab Galaxy instance (http://huttenhower.sph.harvard.edu/galaxy/). PCoA, PERMANOVA and MaAsLin2 analysis are implemented in the vegan R package V.2.5-7. Benjamini-Hochberg correction was used to control for multiple testing, and results were considered significant at FDR <0.05.

### Public Data Download and Processing

1597 raw shotgun stool metagenomes were acquired from 12 independently published studies across 11 countries (114 for UC [24-26], 102 for CD [25-27], 218 for CVD [8], 177 for CRC [28-30], 86 for adenoma [29,30], 83 for IBS-D [31-33], 81 for Obesity [34], and corresponding 736 health controls). The public available raw sequencing data were downloaded through the Sequence Read Archive using the retrieved accession numbers from cited papers. After downloading, the quality filtration and species-level taxonomic profiling were performed according to the above process.

### Data Availability

The raw microbiome sequencing data is publicly available in the Sequence Read Archive under BioProject accession: PRJNA841786. All analyzed or phenotype data can be accessed by appropriate request to the corresponding author (S.C.N) after verifying whether the request is subject to any patients' confidentiality obligation. The analyzed and phenotype data can only be requested for research/scientific purposes to comply with the informed consent signed by study participants, which specifies that the collected data will not be used for commercial purposes. Submitted request will be additionally evaluated by the CU-Med Biobank, and a response to requests will be given within four weeks. All other data supporting the findings of this study are available within the paper and its supplementary files.

### Code Availability

Open-source codes and scripts used for the microbiome analyses or figures are available at the GitHub repository (https://github.com/qsu123/multi-class-classificer). All machine learning data processing and modeling were conducted on Python 3.6.7 using standard libraries that are publicly available: pandas (0.23.4), numpy (1.14.5), scikit-learn (1.1), and matplotlib (2.2.3). The developed multi-class algorithms and trained models can be obtained after appropriate request to the corresponding author (S.C.N) with written non-commercial use statement.

### IV. Predicting Health Conditions

### A. Training a Model to Predict Health Conditions

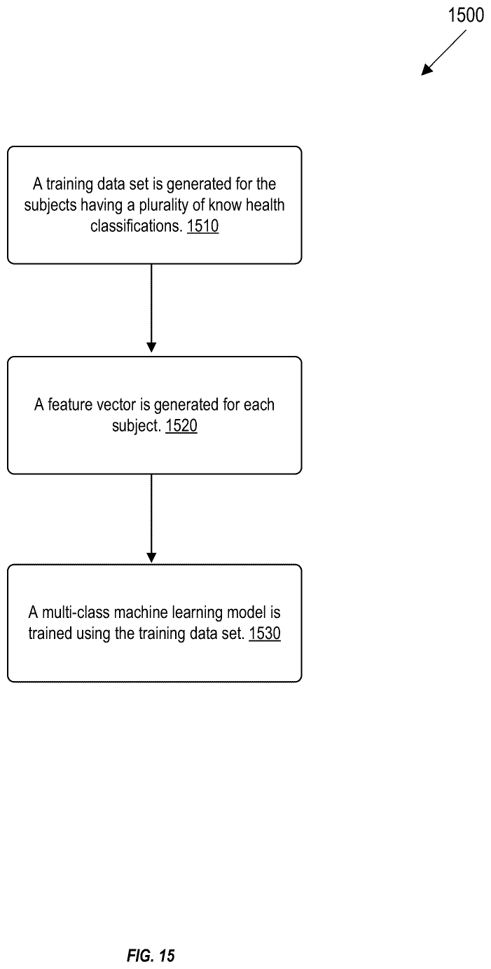

FIG. 15 is a diagram of a method 1500 for training a model to predict health conditions according to an embodiment. Method 1500 can train a multi-class machine learning model to determine risks (e.g., probabilities) of multiple conditions in a subject. Method 1500 and any of the methods described herein may be totally or partially performed with a computer system including one or more processors, which can be configured to perform the steps. Thus, embodiments are directed to computer systems configured to perform the steps of any of the methods described herein, potentially with different components performing a respective step or a respective group of steps.

At block 1510, a training data set is generated for the subjects having a plurality of known health classifications. The training data set can be generated for each cohort of M subjects. As examples, the health classifications (e.g., health conditions) can include Post-acute COVID-19 syndrome (PACS), Crohn's disease (CD), colorectal cancer (CRC), colorectal adenoma (CA), obesity (Ob), and cardiovascular disease (CVD).

For each cohort of M cohorts of subjects, a relative abundance of DNA fragments corresponding to each bacterial species of N bacterial species can be measured in a sample of each of the subjects. N can be ten or more, e.g., 10, 11, 12, 13, 14, 15, 16, 17, 18, 19, 20, 25, 30, 35, 40, 45, 50, 60, 70, 80, 90, or 100, or other numbers described herein. Other values can also be used, such as 5, 6, 7, 8, or 9. Each subject in a cohort has a health classification such that the M cohorts correspond to M health classifications. M can being three or more, e.g., 3, 4, 5, 6, 7, 8, 9, 10, or other values described herein. The M health classifications include healthy and a plurality of conditions. At least ten of the N bacterial species were present in greater than a specified percentage of the subjects, e.g., as described below.

The training data set can be generated by measuring the relative abundance of deoxyribonucleic acid (DNA) fragments in a sample. The DNA can correspond to the bacterial species in the sample, and the sample can be a fecal sample from each subject in a cohort. Other samples can be used such as gut mucosal samples. There can be three or more cohorts where each cohort contains ten or more subjects and a cohort corresponds to subjects (e.g., individuals) with a health classification (e.g., health condition). At least 20 of the bacterial species in the sample can be present in at least a specified percentage (e.g., 5%, 10%, 15%, 20%, 25%, 50%, 75% or more of the subjects), and a species can be present if the relative abundance for the species is greater than 0.01%, 0.05%, 0.10%, 0.15%, 0.20%, 0.50%, 1.00% or more.

The bacterial species can be selected from a list of species such as those listed in Table 1, Table 2, Table 3, FIG. 3D, FIG. 13C, or FIG. 14. In one embodiment, the bacterial species include Blautia_wexlerae, Fusicatenibacter_saccharivorans, Bacteroides_vulgatus, Agathobaculum_butyriciproducens, and Dorea_longicatena. The bacterial species can include the bacterial species having the top 5, 6, 7, 8, 9, 10, 11, 12, 13, 14, 15, 16, 17, 18, 19, 20, 25, 30, 35, 40, 45, 50, 60, 70, 80, 90, or 100 scores from Table 1.

Measuring the relative abundance of the DNA fragments for each subject can include receiving a sequence of reads from sequencing the fecal sample and may include the sequencing itself. A gut mucosal sample can be sequenced in some circumstances. The sequence reads can be aligned to a human reference genome and a database of bacterial genomes, each corresponding to a different bacterial species. The relative abundance can for each of the bacterial species can be determined using the sequence reads corresponding to the bacterial reference genomes.

As another example, measuring the relative abundance of the DNA fragments for each subject can include using a probe-based technique. A probe-based technique can be used to measure the number of DNA fragments of selected features and total number of DNA fragments. A signal can be provided for a particular probe, where the signal (e.g., an intensity signal or a digital signal indicating presence or absence) can provide a number of DNA fragments corresponding to a particular probe. The relative abundance of each feature can be calculated by above numbers.

At block 1520, a feature vector can be generated for each subject. The feature vector can contain the relative abundances of the bacterial species in the subject's sample. The feature vector can be an N dimensional vector where N is a number of bacterial species. The relative abundance for a species can be the number of that species of bacteria divided by the total number of bacteria in the sample (e.g., the percent of a particular bacteria compared to the total number of bacteria in the sample).

At block 1530, a multi-class machine learning model can be trained using the training data set. The machine learning model can be a random forest (RF) model, k nearest neighbors model (KNN), multilayer perceptron model (MLP), or a support vector machine (SVM). The multi-class machine learning model can provide a probability for each of the M health classifications (e.g., health conditions) and the training data set can include the known health classifications for the subjects and the subject's feature vectors. The model can be trained from an algorithm by optimizing the sensitivity and specificity for determining correct conditions by comparing the highest probability to a threshold corresponding to the correct health condition. The training can optimize a sensitivity and a specificity for determining correct conditions (i.e., output of model matches the known health classification) by achieving a highest average AUC of the M health classifications.

An individual may be categorized as having a health condition if the probability for the health classification is the highest probability or if the probability above a threshold. An individual who has been classified with a health classification can be treated for that condition. Treatment can include modifying an existing course of treatment or treatments to modify the fecal microbiome (e.g., fecal transplantation, probiotics, etc.).

B. Predicting Health Conditions with a Trained Model

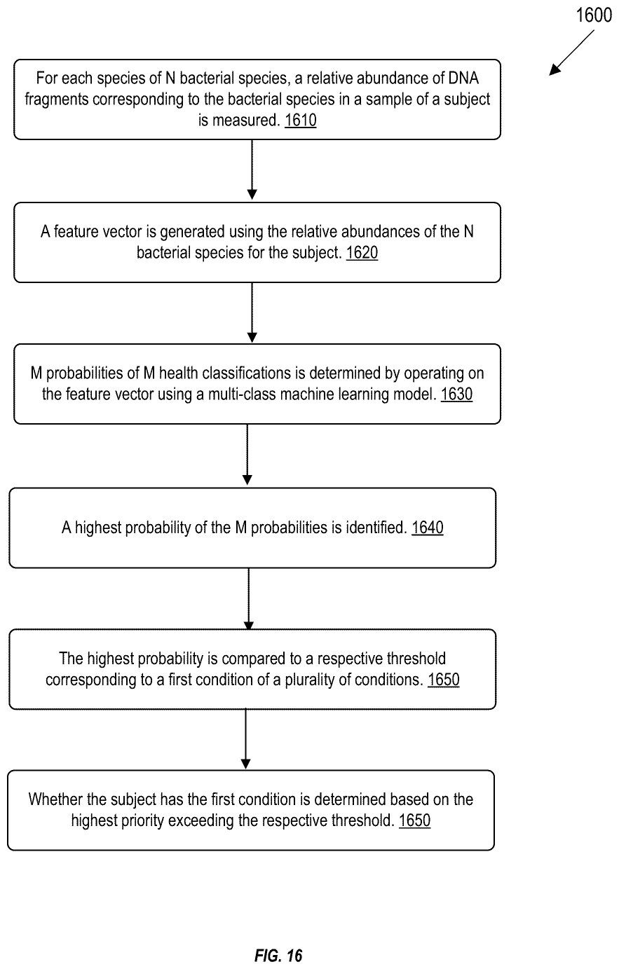

FIG. 16 is a diagram of a method 1600 for predicting health conditions according to an embodiment. Method 1600 can implement a multi-class machine learning model to discriminate among multiple possible conditions of a subject. Method 1600 and any of the methods described herein may be totally or partially performed with a computer system including one or more processors, which can be configured to perform the steps. Thus, embodiments are directed to computer systems configured to perform the steps of any of the methods described herein, potentially with different components performing a respective step or a respective group of steps. Method 1600 can be performed using techniques described herein.

At block 1610, a relative abundance of DNA fragments can be measured in a sample of a subject. The DNA fragments can correspond to bacterial species in the sample, and the relative abundance can be measured for each species of N bacterial species. N can be ten or more, e.g., 10, 11, 12, 13, 14, 15, 16, 17, 18, 19, 20, 25, 30, 35, 40, 45, 50, 60, 70, 80, 90, or 100, or other numbers described herein. Other values can also be used, such as 5, 6, 7, 8, or 9.

The DNA can correspond to the bacterial species in the sample, and the sample can be a fecal sample from each subject in a cohort. Other samples can be used such as gut mucosal samples. There can be three or more cohorts where each cohort contains ten or more subjects and a cohort corresponds to subjects (e.g., individuals) with a health classification (e.g., health condition). At least 20 of the bacterial species in the sample can be present in at least a specified percentage (e.g., 5%, 10%, 15%, 20%, 25%, 50%, 75% or more of the subjects), and a species can be present if the relative abundance for the species is greater than 0.01%, 0.05%, 0.10%, 0.15%, 0.20%, 0.50%, 1.00% or more.

The bacterial species can be selected from a list of species such as those listed in Table 1, Table 2, Table 3, FIG. 3D, FIG. 13C, or FIG. 14. In one embodiment, the bacterial species include Blautia_wexlerae, Fusicatenibacter_saccharivorans, Bacteroides_vulgatus, Agathobaculum_butyriciproducens, and Dorea_longicatena. The bacterial species can include the bacterial species having the top 5, 6, 7, 8, 9, 10, 11, 12, 13, 14, 15, 16, 17, 18, 19, 20, 25, 30, 35, 40, 45, 50, 60, 70, 80, 90, or 100 scores from Table 1.

Measuring the relative abundance of the DNA fragments for each subject can include receiving a sequence of reads from sequencing the fecal sample and may include the sequencing itself. A gut mucosal sample can be sequenced in some circumstances. The sequence reads can be aligned to a human reference genome and a database of bacterial genomes, each corresponding to a different bacterial species. The relative abundance can for each of the bacterial species can be determined using the sequence reads corresponding to the bacterial reference genomes.

As another example, measuring the relative abundance of the DNA fragments for each subject can include using a probe-based technique. A probe-based technique can be used to measure the number of DNA fragments of selected features and total number of DNA fragments. A signal can be provided for a particular probe, where the signal (e.g., an intensity signal or a digital signal indicating presence or absence) can provide a number of DNA fragments corresponding to a particular probe. The relative abundance of each feature can be calculated by above numbers.

At block 1620, a feature vector can be generated using the relative abundances of the N bacterial species for the subject. The N bacterial species for the subject can be the bacterial species identified in a sample derived from the subject. The feature vector can contain the relative abundances of the bacterial species in the subject's sample. The feature vector can be an N dimensional vector where N is a number of bacterial species. The relative abundance for a species can be the number of that species of bacteria divided by the total number of bacteria in the sample (e.g., the percent of a particular bacteria compared to the total number of bacteria in the sample).

At block 1630, M probabilities of M health classifications can be determined by operating on the feature vector using a multi-class machine learning model. M can being three or more, e.g., 3, 4, 5, 6, 7, 8, 9, 10, or other values described herein. As examples, the health classifications (e.g., health conditions) can include Post-acute COVID-19 syndrome (PACS), Crohn's disease (CD), colorectal cancer (CRC), colorectal adenoma (CA), obesity (Ob), and cardiovascular disease (CVD).

The machine learning model can be a random forest (RF) model, k nearest neighbors model (KNN), multilayer perceptron model (MLP), or a support vector machine (SVM). The multi-class machine learning model can provide a probability for each of the health classifications (e.g., health conditions) and the training data set can include the known health classifications for the subjects and the subject's feature vectors. The model can be trained from an algorithm by optimizing the sensitivity and specificity for determining a correct health condition by comparing the highest probability to a threshold corresponding to the correct health condition.

At block 1640, a highest probability of the M probabilities can be identified. An individual may be categorized as having a health condition if the probability for the health classification is the highest probability or if the probability above a threshold. An individual who has been classified with a health classification can be treated for that condition. Treatment can include modifying an existing course of treatment or treatments to modify the fecal microbiome (e.g., fecal transplantation, probiotics, etc.).

At block 1650, the highest probability can be compared to a respective threshold corresponding to a first condition of a plurality of conditions. The M probabilities can be compared to respective thresholds corresponding to the plurality of conditions. Whether the subject has multiple conditions can be determined based on the probabilities exceeding the respective thresholds.

At block 1660, whether the subject has the first condition can be determined based on the highest priority exceeding the respective threshold. The subject may be treated for the condition. For example, the subject can be treated with a microbial treatment (e.g., microbial transplant). The microbiral treatment can be selected from the list disclosed in Table 4.

### V. Example Systems

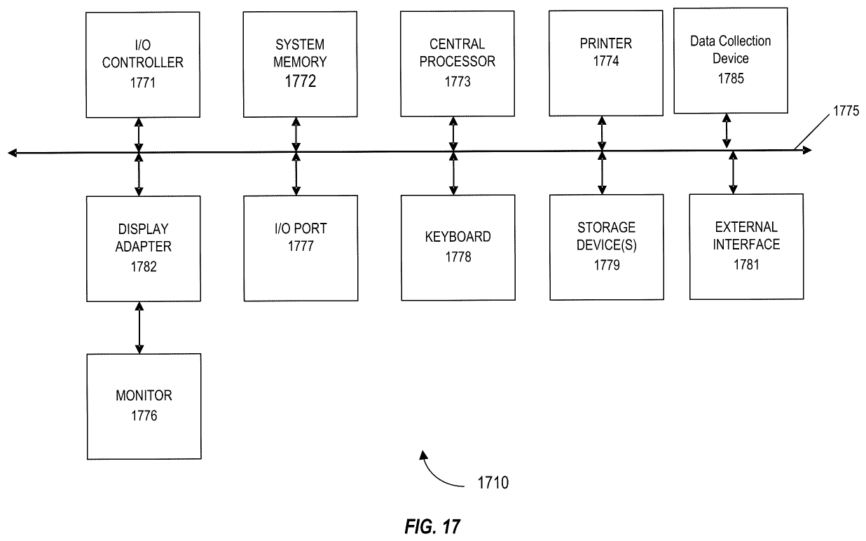

Any of the computer systems mentioned herein may utilize any suitable number of subsystems. Examples of such subsystems are shown in FIG. 17 in computer system 1710. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 17 are interconnected via a system bus 1775. Additional subsystems such as a printer 1774, keyboard 1778, storage device(s) 1779, monitor 1776 (e.g., a display screen, such as an LED), which is coupled to display adapter 1782, and others are shown. Peripherals and input/output (I/O) devices, which couple to I/O controller 1771, can be connected to the computer system by any number of means known in the art such as input/output (I/O) port 1777 (e.g., USB, FireWire®). For example, I/O port 1777 or external interface 1781 (e.g. Ethernet, Wi-Fi, etc.) can be used to connect computer system 1710 to a wide area network such as the Internet, a mouse input device, or a scanner. The interconnection via system bus 1775 allows the central processor 1773 to communicate with each subsystem and to control the execution of a plurality of instructions from system memory 1772 or the storage device(s) 1779 (e.g., a fixed disk, such as a hard drive, or optical disk), as well as the exchange of information between subsystems. The system memory 1772 and/or the storage device(s) 1779 may embody a computer readable medium. Another subsystem is a data collection device 1785, such as a camera, microphone, accelerometer, and the like. Any of the data mentioned herein can be output from one component to another component and can be output to the user.

A computer system can include a plurality of the same components or subsystems, e.g., connected together by external interface 1781, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g. an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

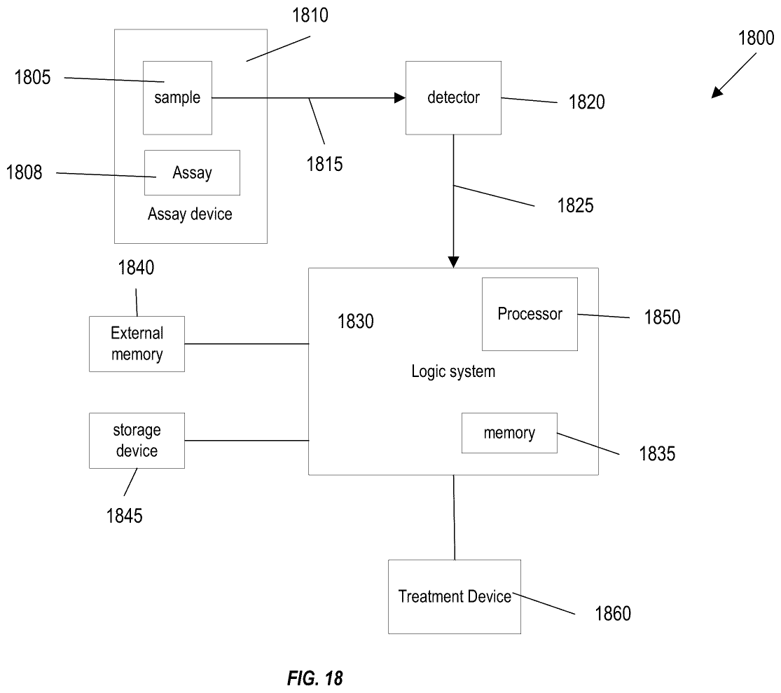

FIG. 18 illustrates a measurement system 1800 according to an embodiment of the present disclosure. The system as shown includes a sample 1805, such as cell-free DNA molecules within an assay device 1810, where an assay 1808 can be performed on sample 1805. For example, sample 1805 can be contacted with reagents of assay 1808 to provide a signal of a physical characteristic 1815. An example of an assay device can be a flow cell that includes probes and/or primers of an assay or a tube through which a droplet moves (with the droplet including the assay). Physical characteristic 1815 (e.g., a fluorescence intensity, a voltage, or a current), from the sample is detected by detector 1820. Detector 1820 can take a measurement at intervals (e.g., periodic intervals) to obtain data points that make up a data signal. In one embodiment, an analog-to-digital converter converts an analog signal from the detector into digital form at a plurality of times. Assay device 1810 and detector 1820 can form an assay system, e.g., a sequencing system that performs sequencing according to embodiments described herein. A data signal 1825 is sent from detector 1820 to logic system 1830. As an example, data signal 1825 can be used to determine sequences and/or locations in a reference genome of DNA molecules. Data signal 1825 can include various measurements made at a same time, e.g., different colors of fluorescent dyes or different electrical signals for different molecule of sample 1805, and thus data signal 1825 can correspond to multiple signals. Data signal 1825 may be stored in a local memory 1835, an external memory 1840, or a storage device 1845.

Logic system 1830 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 1830 and the other components may be part of a stand-alone or network connected computer system, or they may be directly attached to or incorporated in a device (e.g., a sequencing device) that includes detector 1820 and/or assay device 1810. Logic system 1830 may also include software that executes in a processor 1850. Logic system 1830 may include a computer readable medium storing instructions for controlling measurement system 1800 to perform any of the methods described herein. For example, logic system 1830 can provide commands to a system that includes assay device 1810 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay.

System 1800 may also include a treatment device 1860, which can provide a treatment to the subject, e.g., as selected from table 4. Treatment device 1860 can determine a treatment and/or be used to perform a treatment. Examples of such treatment can include histamine antagonists physical therapy, counseling, breathing exercise, pulmonary and cardiac rehabilitation, memory exercises, olfactory training for post-acute COVID-19 syndrome; anti-inflammatory drugs (e.g. 5-aminosalicylic acid), immunosuppressant (e.g. azathioprine, mercaptopurine, methotrexate, cyclosporine), biologics (e.g. tumor necrosis factor-alpha blockers, integrin blockers, interleukin blockers) and surgery for Crohn's disease and ulcerative colitis; surgery, radiation therapy, chemotherapy, immunotherapy, targeted therapy, hormone therapy, and stem cell transplant for colorectal cancer and colorectal adenoma; calorie-controlled diet, exercise, naltrexone/bupropion, phentermine/topiramate ER, glucagon-like peptide-1 receptor agonists, sodium-glucose cotransporter-2 inhibitor and bariatric surgery for obesity; low-dose aspirin, clopidogrel, rivaroxaban, ticagrelor, prasugrel, statin (e.g. atorvastatin, simvastatin, rosuvastatin, pravastatin), beta blockers (e.g. atenolol, bisoprolol, metoprolol), nitrates, angiotensin-converting enzyme inhibitors (e.g. ramipril, lisinopril), angiotensin-2 receptor blockers, calcium channel blockers (e.g. amlodipine, verapamil), diuretics, coronary angioplasty, coronary artery bypass grafting for cardiovascular disease; dietary modification, smooth muscle relaxants, low-dose antidepressants, psychotherapy for IBS. Such treatment can also include microbiome modulation by supplementation with probiotics, prebiotics or synbiotics, or through modification of diet. Logic system 1830 may be connected to treatment device 1860, e.g., to provide results of a method described herein. The treatment device may receive inputs from other devices, such as an imaging device and user inputs (e.g., to control the treatment, such as controls over a robotic system).

Any of the methods described herein may be totally or partially performed with a computer system including one or more processors, which can be configured to perform the steps. Any operations performed with a processor (e.g., aligning, determining, comparing, computing, calculating) may be performed in real-time. The term “real-time” may refer to computing operations or processes that are completed within a certain time constraint. The time constraint may be 1 minute, 1 hour, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

All patents, patent applications, publications, and descriptions mentioned herein are incorporated by reference in their entirety for all purposes. None is admitted to be prior art. Where a conflict exists between the instant application and a reference provided herein, the instant application shall dominate.

### REFERENCES

1. Lynch, S. V. & Pedersen, O. The Human Intestinal Microbiome in Health and Disease. New England Journal of Medicine 375, 2369-2379 (2016).
2. Metwaly, A., Reitmeier, S. & Haller, D. Microbiome risk profiles as biomarkers for inflammatory and metabolic disorders. Nat Rev Gastroenterol Hepatol (2022).
3. Lin, Y., Wang, G., Yu, J. & Sung, J. J. Y. Artificial intelligence and metagenomics in intestinal diseases. J Gastroenterol Hepatol 36, 841-847 (2021).
4. Liang, J. Q., et al. A novel fecal Lachnoclostridium marker for the non-invasive diagnosis of colorectal adenoma and cancer. Gut 69, 1248-1257 (2020).
5. Vila, A. V., et al. Gut microbiota composition and functional changes in inflammatory bowel disease and irritable bowel syndrome. Science Translational Medicine 10, eaap8914 (2018).
6. Shaukat, A. & Levin, T. R. Current and future colorectal cancer screening strategies. Nat Rev Gastroenterol Hepatol (2022).
7. Turnbaugh, P. J., et al. An obesity-associated gut microbiome with increased capacity for energy harvest. Nature 444, 1027-1031 (2006).
8. Jie, Z., et al. The gut microbiome in atherosclerotic cardiovascular disease. Nat Commun 8, 845 (2017).
9. Yeoh, Y. K., et al. Gut microbiota composition reflects disease severity and dysfunctional immune responses in patients with COVID-19. Gut 70, 698-706 (2021).
10. Liu, Q., et al. Gut microbiota dynamics in a prospective cohort of patients with post-acute COVID-19 syndrome. Gut 71, 544-552 (2022).
11. Vestad, B., et al. Respiratory dysfunction three months after severe COVID-19 is associated with gut microbiota alterations. J Intern Med 291, 801-812 (2022).
12. Gacesa, R., et al. Environmental factors shaping the gut microbiome in a Dutch population. Nature 604, 732-739 (2022).
13. Gupta, V. K., et al. A predictive index for health status using species-level gut microbiome profiling. Nat Commun 11, 4635 (2020).
14. Wyres, K. L., Lam, M. M. C. & Holt, K. E. Population genomics of Klebsiella pneumoniae. Nat Rev Microbiol 18, 344-359 (2020).
15. Nie, K., et al. Roseburia intestinalis: A Beneficial Gut Organism From the Discoveries in Genus and Species. Front Cell Infect Microbiol 11, 757718 (2021).
16. Margherita Grandini, E. B., Giorgio Visani. Metrics for Multi-Class Classification: an Overview. arXiv (2020).
17. Stojanov, S., Berlec, A. & Strukelj, B. The Influence of Probiotics on the Firmicutes/Bacteroidetes Ratio in the Treatment of Obesity and Inflammatory Bowel disease. Microorganisms 8 (2020).
18. Xu, J., et al. Alteration of the abundance of Parvimonas micra in the gut along the adenoma-carcinoma sequence. Oncol Lett 20, 106 (2020).
19. Lowenmark, T., et al. Parvimonas micra as a putative non-invasive fecal biomarker for colorectal cancer. Sci Rep 10, 15250 (2020).
20. Nalbandian, A., et al. Post-acute COVID-19 syndrome. Nat Med 27, 601-615 (2021).
21. Chen, Z., et al. Impact of Preservation Method and 16S rRNA Hypervariable Region on Gut Microbiota Profiling. mSystems 4, e00271-00218 (2019).
22. Pedregosa, F., et al. Scikit-learn: Machine learning in Python. the Journal of machine Learning research 12, 2825-2830 (2011).
23. Pasolli, E., Truong, D. T., Malik, F., Waldron, L. & Segata, N. Machine Learning Meta-analysis of Large Metagenomic Datasets: Tools and Biological Insights. PLOS Comput Biol 12, e1004977 (2016).
24. Franzosa, E. A., et al. Gut microbiome structure and metabolic activity in inflammatory bowel disease. Nat Microbiol 4, 293-305 (2019).
25. Nielsen, H. B., et al. Identification and assembly of genomes and genetic elements in complex metagenomic samples without using reference genomes. Nat Biotechnol 32, 822-828 (2014).
26. Weng, Y. J., et al. Correlation of diet, microbiota and metabolite networks in inflammatory bowel disease. J Dig Dis 20, 447-459 (2019).
27. He, Q., et al. Two distinct metacommunities characterize the gut microbiota in Crohn's disease patients. Gigascience 6, 1-11 (2017).
28. Yachida, S., et al. Metagenomic and metabolomic analyses reveal distinct stage-specific phenotypes of the gut microbiota in colorectal cancer. Nat Med 25, 968-976 (2019).
29. Feng, Q., et al. Gut microbiome development along the colorectal adenoma-carcinoma sequence. Nat Commun 6, 6528 (2015).
30. Zeller, G., et al. Potential of fecal microbiota for early-stage detection of colorectal cancer. Mol Syst Biol 10, 766 (2014).
31. Vervier, K., et al. Two microbiota subtypes identified in irritable bowel syndrome with distinct responses to the low FODMAP diet. Gut (2021).
32. Goll, R., et al. Effects of fecal microbiota transplantation in subjects with irritable bowel syndrome are mirrored by changes in gut microbiome. Gut Microbes 12, 1794263 (2020).
33. Mars, R. A. T., et al. Longitudinal Multi-omics Reveals Subset-Specific Mechanisms Underlying Irritable Bowel Syndrome. Cell 182, 1460-1473 e1417 (2020).
34. Meslier, V., et al. Mediterranean diet intervention in overweight and obese subjects lowers plasma cholesterol and canses changes in the gut microbiome and metabolome independently of energy intake. Gut 69, 1258-1268 (2020).

What is claimed is:

1. A method of using a multi-class machine learning model to discriminate among multiple possible conditions of a subject, the method comprising:
for each bacterial species of N bacterial species, measuring a relative abundance of DNA fragments corresponding to the bacterial species in a sample of the subject, wherein N is ten or more;
generating a feature vector using the relative abundances of the N bacterial species for the subject;
determining M probabilities of M health classifications by operating on the feature vector using the multi-class machine learning model, M being three or more, wherein the M health classifications include healthy and a plurality of conditions;
identifying a highest probability of the M probabilities;
comparing the highest probability to a respective threshold corresponding to a first condition of the plurality of conditions; and
determining the subject has the first condition based on the highest probability exceeding the respective threshold.

2. The method of claim 1, further comprising:
comparing the M probabilities to respective thresholds corresponding to the plurality of conditions; and
determining the subject has multiple conditions based on the M probabilities exceeding the respective thresholds.

3. The method of claim 1, wherein the sample is a fecal sample or a gut mucosal sample.

4. The method of claim 1, wherein the N bacterial species are selected from Table 1.

5. The method of claim 1, wherein the N bacterial species are selected from Table 2.

6. The method of claim 1, wherein the N bacterial species are selected from Table 3.

7. The method of claim 1, wherein measuring the relative abundance of DNA fragments includes:
for each subject:
receiving a set of sequence reads obtained from a sequencing of the sample; and
aligning the set of sequence reads to a human reference genome and a database of bacterial reference genomes; and
determining the relative abundance for each of the N bacterial species using the sequence reads corresponding to the bacterial reference genomes.

8. The method of claim 1, wherein measuring the relative abundance of DNA fragments uses probes.

9. The method of claim 1, wherein the plurality of conditions include post-acute COVID-19 syndrome (PACS), Crohn's disease (CD), ulcerative colitis (UC), colorectal cancer (CRC), colorectal adenoma (CA), obesity (Ob), diarrhea-dominant irritable bowel syndrome (IBS-D) and cardiovascular disease (CVD).

10. The method of claim 1, wherein the multi-class machine learning model includes random forest, K-nearest neighbors, multi-layer perceptron, graph convolutional neural network, or support vector machine.

11. A method of training a multi-class machine learning model to determine risks of multiple conditions in a subject, the method comprising:
generating a training data set for subjects having a plurality of known health classifications by:
for each cohort of M cohorts of subjects:
for each bacterial species of N bacterial species, measuring a relative abundance of DNA fragments corresponding to the bacterial species in a sample of each of the subjects,
wherein each subject in a cohort has a health classification such that the M cohorts correspond to M health classification, M being three or more, wherein the M health classifications include healthy and a plurality of conditions, wherein N is ten or more, and
wherein at least ten of the N bacterial species were present in greater than a specified percentage of the subjects;
for each subject, generating a feature vector using the relative abundances of the N bacterial species for the subject; and
training the multi-class machine learning model using the training data set, including the known health classifications for the subjects and the feature vectors for the subjects, wherein the multi-class machine learning model provides a probability for each of the M health classifications, and wherein the training optimizes sensitivity and specificity for determining correct conditions by achieving a highest average AUC of the M health classifications.

12. The method of claim 11, wherein the specified percentage is at least 5%.

13. The method of claim 11, wherein the sample of each of the subjects is a fecal sample or a gut mucosal sample.

14. The method of claim 11, wherein the N bacterial species are selected from Table 1.

15. The method of claim 11, wherein the N bacterial species are selected from Table 2.

16. The method of claim 11, wherein the N bacterial species are selected from Table 3.

17. The method of claim 11, wherein measuring the relative abundance of DNA fragments includes:
for each subject:
receiving a set of sequence reads obtained from a sequencing of the sample; and
aligning the set of sequence reads to a human reference genome and a database of bacterial reference genomes; and
determining the relative abundance for each of the N bacterial species using the sequence reads corresponding to the bacterial reference genomes.

18. The method of claim 11, wherein measuring the relative abundance of DNA fragments uses probes.

19. The method of claim 11, wherein the plurality of conditions include post-acute COVID-19 syndrome (PACS), Crohn's disease (CD), ulcerative colitis (UC), colorectal cancer (CRC), colorectal adenoma (CA), obesity (Ob), diarrhea-dominant irritable bowel syndrome (IBS-D) and cardiovascular disease (CVD).

20. A computer product comprising a non-transitory computer readable medium storing a plurality of instructions that, when executed, control a computer system to perform operations to use a multi-class machine learning model to discriminate among multiple possible conditions of a subject by:
for each bacterial species of N bacterial species, measuring a relative abundance of DNA fragments corresponding to the bacterial species in a sample of the subject, wherein N is ten or more;
generating a feature vector using the relative abundances of the N bacterial species for the subject;
determining M probabilities of M health classifications by operating on the feature vector using the multi-class machine learning model, M being three or more, wherein the M health classifications include healthy and a plurality of conditions;
identifying a highest probability of the M probabilities;
comparing the highest probability to a respective threshold corresponding to a first condition of the plurality of conditions; and
determining the subject has the first condition based on the highest probability exceeding the respective threshold.
